(19) United States
(12) Patent Application Publication
(10) Pub. No.: US 2021/0177917 A1
(43) Pub. Date: Jun. 17, 2021

(54) MOLECULAR BACTERIOTHERAPY TO CONTROL SKIN ENZYMATIC ACTIVITY

(71) Applicant: The Regents of the University of California, Oakland, CA (US)

(72) Inventors: Richard L. Gallo, San Diego, CA (US); Michael Williams, San Diego, CA (US)

(21) Appl. No.: 16/642,340

(22) PCT Filed: Aug. 31, 2018
(86) PCT No.: PCT/US2018/049237

§ 371 Date: Feb. 26, 2020

Related U.S. Application Data
(60) Provisional application No. 62/553,025, filed on Aug. 31, 2017.

(51) Int. Cl. A61K 35/741; A61K 9/00; A61K 38/08; C12Q 1/37; A61K 9/06; A61P 17/00

(52) U.S. Cl. CPC A61K 35/741; A61K 9/0014; A61K 38/08; G01N 2800/202; A61K 9/06; A61P 17/00; G01N 2333/96425; C12Q 1/37

(57) ABSTRACT

The disclosure relates to composition and methods to treat dermatological diseases and disorders and to composition that modulate skin barrier permeability.

### CROSS REFERENCE TO RELATED APPLICATIONS

This application claims priority under 35 U.S.C. § 119 from Provisional Application Ser. No. 62/553,025, filed Aug. 31, 2017, the disclosures of which are incorporated herein by reference.

### TECHNICAL FIELD

The disclosure relates to composition and methods to treat dermatological diseases and disorders and to composition that modulate skin barrier permeability.

### Microorganism Deposit

Exemplary microorganisms of the disclosure (Staphylococcus epidermidis A11, Staphylococcus hominis C5, Staphylococcus hominis A9 and Staphylococcus warneri G2) were deposited on Aug. 28, 2018 with the American Type Culture Collection, 10801 University Boulevard, Manassas, Va. 20110-2209, as ATCC Number ______ (strain designation S. epidermidis A11 81618, deposited Aug. 28, 2018), ATCC Number ______ (strain designation S. hominis C5 81618, deposited Aug. 28, 2018), ATCC Number ______ (strain designation S. hominis A9 81618, deposited Aug. 28, 2018) and as ATCC Number ______ (strain designation S. warneri G2 81618, deposited Aug. 28, 2018) under the Budapest Treaty. This deposit will be maintained at an authorized depository and replaced in the event of mutation, non-viability or destruction for a period of at least five years after the most recent request for release of a sample was received by the depository, for a period of at least thirty years after the date of the deposit, or during the enforceable life of the related patent, whichever period is longest. All restrictions on the availability to the public of these cell lines will be irrevocably removed upon the issuance of a patent from the application.

### STATEMENT AS TO FEDERALLY SPONSORED RESEARCH

This invention was made with Government support under Grant Nos. AI117673, AR067547, AR062496, and AR064781 awarded by the National Institutes of Health. The Government has certain rights in the invention.

### BACKGROUND

The epidermis is the first line of immune defense and protects and regulates interactions between microbes and the host organism. Control of this interaction is important because bacteria not only reside on the surface where they influence superficial keratinocytes, but also penetrate below the stratum corneum and into the dermis where some bacterial species have been shown to influence immune function. For example, Staphylococcus epidermidis (S. epidermidis) interacts with epidermal keratinocytes to prevent toll-like receptor 3-mediated inflammation, recruits mast cells and T cells, and increases tight junctions and antimicrobial peptide production. In contrast to the common skin commensal bacteria, S. epidermidis, Staphylococcus aureus (S. aureus) is often pathogenic and has a negative influence on skin function. This is especially evident in skin diseases such as atopic dermatitis (AD) where S. aureus promotes this disease.

The microbiome inhabiting the skin of subjects with AD has been shown to have a decrease in overall microbial diversity and an increase in S. aureus abundance. Increased S. aureus colonization has been linked to increased disease severity for patients with AD. Mechanistically, it is unclear how S. aureus worsens disease. Several products from S. aureus have been shown to damage the barrier and/or trigger inflammation. These products include a-toxin, superantigens, toxic shock syndrome toxin 1, enterotoxins, protein A, Panton-Valentine leukocidin, exfoliative toxins, and V8 serine Protease. Because of the potential pathogenic effects of these molecules, understanding the response of the skin to S. aureus colonization in the absence of clear clinical signs of infection is critical to understanding the pathogenesis of AD and for developing future therapies.

### SUMMARY

The disclosure provides a purified polypeptide comprising a sequence that is at least 98% identical to SEQ ID NO:4, 11, 12, 13, 14, 15, 16, or 17 and which inhibits (i) protease production and/or activity of keratinocytes, (ii) inhibits IL-6 production and/or activity of keratinocytes, (iii) inhibits production of phenol soluble modulin alpha 3 from Staphylococcus aureus (S. aureus) and/or (iv) inhibits agr production and/or activity by S. aureus. In one embodiment, the polypeptide is at least 98% identical to SEQ ID NO:2. In another embodiment the polypeptide comprises SEQ ID NO:4, 11, 12, 13, 14, 15, 16, or 17. In yet another embodiment, the polypeptide consists of SEQ ID NO:4, 11, 12, 13, 14, 15, 16, or 17. In another or further embodiment of any of the foregoing, the polypeptide comprises one or more D-amino acids. In yet another or further embodiment, the polypeptide comprises a compound of Formula I, IA or IB (see below).

The disclosure also provides a topical formulation comprising a polypeptide of the disclosure or a compound for Formula I, IA or IB.

The disclosure also provides an isolated polynucleotide encoding a polypeptide of the disclosure. In one embodiment, the polynucleotide comprises a sequence that hybridizes under stringent conditions to a polynucleotide consisting of SEQ ID NO:1 or 3 and encodes a polypeptide comprising SEQ ID NO:4. In another embodiment, the polynucleotide comprises SEQ ID NO:1 or 3.

The disclosure also provides vectors comprising a polynucleotide of the disclosure. The vector can be any suitable vector for expression in a cell or microbial host.

The disclosure also provides a recombinant microorganism comprising a vector or polynucleotide of the disclosure. In some embodiments, the microorganism does not naturally express a polypeptide of the disclosure by through recombinant engineering is engineered to expression a polynucleotide of the disclosure. In still another embodiment, the microorganism is attenuated in that it has been rendered non-pathogenic or has reduced pathogenicity compared to a wild-type organism of the same species. In still another embodiment, the recombinant microorganism is an microorganism normally found (e.g. commensal) to the skin of the mammal (e.g., a human).

The disclosure also provide a probiotic composition comprising a recombinant microorganism of the disclosure.

The disclosure also provides a probiotic composition comprising a microorganism that expresses a polypeptide of the disclosure (e.g., SEQ ID NO:4, 11, 12, 13, 14, 15, 16, and/or 17). In one embodiment, the microorganism is S. hominis, S. epidermidis, S. warneri or any combination thereof. In a further embodiment, the microorganism is S. hominis C5, S. hominis A9, S. epidermidis A11 and/or S. warneri G2. In yet another or further embodiment, the composition comprises a microorganism selected from the group of microorganisms having ATCC Number ______ (strain designation S. epidermidis A11 81618, deposited Aug. 28, 2018), ATCC Number ______ (strain designation S. hominis C5 81618, deposited Aug. 28, 2018), ATCC Number ______ (strain designation S. hominis A9 81618, deposited Aug. 28, 2018), ATCC Number (strain designation S. warneri G2 81618, deposited Aug. 28, 2018) and any combination of the foregoing strains. In another embodiment, the probiotic composition of the disclosure is non-natural (e.g., is does not include the full spectrum of microorganism found on the skin, or includes amounts of microorganisms per unit volume that are not found on the skin, or the microorgnaisms have been genetically modified, or the composition contains components or compounds that are not normally found on the skin).

The disclosure also provides a method of treating a dermatological disorder comprising administering an effective amount of a coagulase negative Staphylococcus sp. (CoNS), or an effective amount of a fermentation extract of CoNS sufficient to inhibit protease activity on the skin, wherein the CoNS produces polypeptide comprising a sequence that is at least 98% identical to SEQ ID NO:4, 11, 12, 13, 14, 15, 16, of 17 and which inhibits protease production. In one embodiment, the dermatological disorders is selected from the group consisting of Netherton syndrome, atopic dermatitis, contact dermatitis, eczema, psoriasis, acne, epidermal hyperkeratosis, acanthosis, epidermal inflammation, dermal inflammation and pruritus. In another embodiment, the administering is by topical application. In still another or further embodiment, the CoNS is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus Hominis, Staphylococcus jettensis, Staphylococcus petrasii, and Staphylococcus lugdunensis. In still another or further embodiment of any of the foregoing, the fermentation extract of the CoNS comprises a polypeptide sequence of SEQ ID NO:4 and/or a compound of Formula I, IA, or IB. In another embodiment, the CoNS is selected from the group consisting of S. epidermidis A11, S. hominis C4, S. hominis C5, S. hominis A9, S. warneri G2 and any combination thereof.

The disclosure also provides a method of treating a skin disease or disorder, comprising measuring the protease activity of a culture from skin of a subject or of skin from the subject; comparing the protease activity to a normal control; administering a commensal skin bacterial composition and/or fermentation extract from a coagulase negative Staphylococci, wherein the commensal skin bacteria composition or fermentation extract comprises a polypeptide that is at least 98% identical to SEQ ID NO:4, 11, 12, 13, 14, 15, 16, or 17, and/or comprises a compound of Formula I, IA or IB, wherein the composition is formulated in a cream, ointment or pharmaceutical composition that maintain the commensal skin bacteria's ability to grow and replicate. In one embodiment, the coagulase negative Staphylococci is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus Hominis, Staphylococcus jettensis, Staphylococcus petrasii, and Staphylococcus lugdunensis.

The disclosure also provides a method of treating a skin disease or disorder comprising administering a purified polypeptide of the disclosure or a probiotic composition comprising a bacteria that produces a polypeptide that is at least 98% identical to SEQ ID NO:4, 11, 12, 13, 14, 15, 16, or 17 and that inhibits kallikrein production or activity.

The disclosure also provides a method of treating a skin disease or disorder comprising administering composition that inhibits phenol soluble modulin expression, wherein the composition comprises a purified polypeptide of the disclosure or a compound of Formula I, IA, or IB. In one embodiment, the administering is topical. In another embodiment, the composition is a fermentation extract of a coagulase negative Staphylococci.

The disclosure also provides a topical probiotic composition comprising a probiotic commensal skin bacteria selected from the group consisting of S. epidermidis A11, S. hominis C4, S. hominis C5, S. hominis A9, S. warneri G2 and any combination thereof. In one embodiment, the composition is formulated as a lotion, shake lotion, cream, ointment, gel, foam, powder, solid, paste or tincture.

The disclosure also provides a drug composition comprising a drug and an S. aureus fermentation extract or S. aureus-probiotic comprising a soluble phenol modulin alpha 3. The disclosure also provides for the use of the composition for delivering a drug through the skin of a subject.

The disclosure provides commensal/good bacteria and/or their products to prevent increased protease activity in the skin. This is important in many disease states including atopic dermatitis, Netherton syndrome and other skin conditions that suffer from abnormally high protease activity and barrier breakdown.

This disclosure also provides factor and compositions to induce protease activity and therefore help with proteolytic remodeling of the skin in treatment of disorders related to wound repair, aging, sun damage, pigment abnormalities and scarring.

The disclosure provides a method of treating a dermatological disorder comprising administering an effective amount of a coagulase negative Staphylococcus sp. (CoNS), or an effective amount of a fermentation extract of CoNS sufficient to inhibit protease activity on the skin. In one embodiment, the dermatological disorders is selected from the group consisting of Netherton syndrome, atopic dermatitis, contact dermatitis, eczema, psoriasis, acne, epidermal hyperkeratosis, acanthosis, epidermal inflammation, dermal inflammation and pruritus. In another embodiment, the administering is by topical application. In another embodiment, the CoNS is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus hominis, Staphylococcus jettensis, Staphylococcus petrasii, and Staphylococcus lugdunensis. In a specific embodiment, the CoNS is S. epidermidis.

The disclosure also provides a method of treating a skin disease or disorder, comprising measuring the protease activity of a culture from skin of a subject or of skin from the subject; comparing the protease activity to a normal control; administering a commensal skin bacterial composition and/or fermentation extract from a coagulase negative Staphylococci, wherein the commensal skin bacterial composition comprises at least one commensal bacteria that reduces serine protease activity of the culture or skin, wherein the at least one commensal bacteria is formulated in a cream, ointment or pharmaceutical composition that maintain the commensal skin bacteria's ability to grow and replicate. In one embodiment, the coagulase negative Staphylococci is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus hominis, Staphylococcus jettensis, Staphylococcus petrasii, and Staphylococcus lugdunensis.

The disclosure also provides a method of treating a skin disease or disorder comprising administering an agent that inhibits kallikrein expression. The disclosure also provides a method of treating a skin disease or disorder comprising administering an agent that inhibits phenol soluble modulin expression. In one embodiment of either of the foregoing, the administering is topical. In another embodiment, the agent is a fermentation extract of a coagulase negative Staphylococci. In another embodiment, the coagulase negative Staphylococci is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus hominis, Staphylococcus jettensis, Staphylococcus petrasii, and Staphylococcus lugdunensis.

The disclosure also provides a topical composition comprising a plurality of skin bacteria. In on embodiment, the probiotic commensal skin bacteria is a coagulase negative Staphylococcus species. In another distinct embodiment, the probiotic commensal skin bacteria comprises Staphylococcus aureus. In one embodiment of either of the foregoing embodiments, the bacterial is formulated in cream, lotion, tincture, gel, or other topical formulary wherein the bacteria remains viable.

The disclosure also provides a topical probiotic composition comprising a probiotic commensal skin bacteria fermentation extract, the probiotic commensal skin bacterial fermentation extract obtained from a coagulase negative staphylococcus (CoNS) species. In one embodiment, the CoNS is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus hominis, Staphylococcus jettensis, Staphylococcus petrasii, and Staphylococcus lugdunensis.

In any of the embodiments described a topical probiotic composition of is formulated as a lotion, shake lotion, cream, ointment, gel, foam, powder, solid, paste or tincture.

The disclosure provides a drug composition comprising a drug and an S. aureus fermentation extract or S. aureus-biotic composition.

The disclosure provides a method for drug delivery through the skin comprising contacting the skin with a composition comprising a drug and an S. aureus fermentation extract or S. aureus-biotic composition. In one embodiment, the drug is a topical drug to be absorbed or adsorbed through the skin.

The disclosure also provides a method of delivering a topical drug, the method comprising contacting the skin of a subject with a composition comprising an S. aureus or a fermentation extract of S. aureus for a time and under a dose and conditions to increase permeability of the skin and then contacting the skin with the drug to be delivered.

The disclosure provides a composition comprising a fermentation extract from S. aureus or a lotion, shake lotion, cream, ointment, gel, foam, powder, solid, paste or tincture containing viable S. aureus.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-D shows (A-C) NHEKs were treated for 24 hours with S. aureus (SA; Newman, USA300, 113, SANGER252) and S. epidermidis (ATCC12228, ATCC1457) sterile filtered supernatants and the NHEK conditioned medium was analyzed with specific trypsin-like, elastase-like, and MMP protease substrates. (D) S. aureus (Newman) secreted proteases were analyzed for their influence on trypsin activity. Data represent mean±SEM (n=4) and are representative of at least three independent experiments. One-way ANOVAs (aec) and two-way ANOVAs (d) were used and significance was indicated by *P<0.05, ***P<0.001, ****P<0.0001. ANOVA, analysis of variance; MMP, matrix metalloproteinase; NHEK, normal human epidermal keratinocyte.

FIG. 2A-C shows (A) Total protease activity (5 μg ml BODIPY FL casein) was measured in the NHEK conditioned medium after S. aureus (SA, Newman) supernatant treatment for 0-48 hours, (B) whereas the serine protease inhibitor aprotinin (800 μg ml) was applied to the 24-hour posttreatment conditioned medium. (C) S. aureus (USA300 LAC) WT and protease-null strains were compared for effects on NHEK conditioned medium trypsin activity (Boc-Val-Pro-Arg-AMC, 200 mM). Both two-way ANOVAs (A,B) and one-way ANOVAs (C) were used and significance was indicated by *P<0.05, **P<0.01, ***P<0.001, ****P<0.0001. ANOVA, analysis of variance; NHEK, normal human epidermal keratinocyte; WT, wild type.

FIG. 3A-F shows S. aureus increases KLK expression in human keratinocytes. (A) Relative abundance of KLK mRNA expression in NHEKs after 24-hour S. aureus (SA, Newman) supernatant treatment was analyzed by qPCR. (B-E) KLK5, 6, 13, and 14 were analyzed for fold changes in mRNA expression in NHEKs treated with S. aureus supernatant for 0-48 hours. All mRNA expression levels were normalized with the housekeeping gene, glyceraldehyde-3-phosphate dehydrogenase (GAPDH). (F) NHEK conditioned medium and cell lysates were analyzed for changes in protein expression of KLK5, 6, 13, and 14 by immunoblotting after a 24-hour treatment with SA (Newman) supernatant using both published and predicted molecular weights. The housekeeping gene, a-tubulin, was used as a loading control for cell lysates. Data represent mean±SEM (n=3) and are representative of at least three independent experiments. Two-way ANOVAs (bee) were used and significance was indicated by **P<0.01, ***P<0.001, ****P<0.0001. ANOVA, analysis of variance; KLK, kallikrein; NHEK, normal human epidermal keratinocyte; qPCR, quantitative real-time PCR; SEM, standard error of the mean.

FIG. 4A-D shows multiple KLKs are responsible for S. aureus-induced serine protease activity in human keratinocytes. NHEKs were treated with KLK6, 13, or 14 siRNA (15 nM) before CaCl₂) differentiation and the addition of S. aureus (Newman) supernatant. siRNA scrambled (−) controls 1 and 2 were used at 15 nM and 45 nM, respectively. (A) Conditioned medium was analyzed for changes in trypsin activity (Boc-Val-Pro-Arg-AMC, 200 μM). (B-D) Transcript levels of KLK6, KLK13, and KLK14 were assessed by qPCR and normalized to the housekeeping gene, GAPDH, to confirm siRNA knockdown efficiency. Data represent mean±SEM (n=4) and are representative of at least three independent experiments. One-way ANOVA (a) was used and significance indicated by *P<0.05, **P<0.01, ***P<0.001. ANOVA, analysis of variance; GAPDH, glyceraldehyde-3-phosphate dehydrogenase; KLK, kallikrein; NHEK, normal human epidermal keratinocyte; qPCR, quantitative real-time PCR; siRNA, small interfering RNA; SEM, standard error of the mean.

FIG. 5A-C shows multiple KLKs regulate S. aureus-induced DSG-1 and FLG cleavage in human keratinocytes. NHEKs treated with S. aureus (Newman) supernatant for 24 hours were assessed for changes to (A) desmoglein-1 (DSG-1) and (B) profilaggrin (Pro-FLG) cleavage after siRNA knockdown of KLK6, 13, and 14 (15 nM) by immunoblotting. The housekeeping gene, a-tubulin, was used as a loading control. DSG-1 (full length) and Pro-FLG are indicated by black arrows. (C) Densitometry analysis of both DSG-1 (full length) and Pro-FLG represented by the average number of pixels normalized to a-tubulin (n=1). Immunoblots are representative of at least three independent experiments. KLK, kallikrein; NHEK, normal human epidermal keratinocyte; siRNA, small interfering RNA.

FIG. 6 depicts a method of preparing fermentation extracts and assays for activity.

FIG. 7 shows S. aureus phenol-soluble modulins (PSMs) under control of agr quorum sensing system are responsible for increased keratinocyte serine protease activity.

FIG. 8 shows S. aureus PSMs increase mouse serine protease activity and skin barrier damage.

FIG. 9 shows S. aureus isolates from atopic dermatitis(AD lesional skin can induce serine protease activity in keratinocytes in an agr-type dependent manner.

FIG. 10 shows that coagulase-negative Staphylococci (CoNS) strain ATCC14490 (S. epidermidis) can produce auto-inducing peptide (AIP) to turn off S. aureus agr activity.

FIG. 11 shows the effect of S. aureus and commensal bacteria on serine protease activity in atopic dermatitis.

FIG. 12 shows the effect of S. hominis C5 on S. aureus agr activity.

FIG. 13 shows the effect of various CoNS strains on S. aureus agr activity.

FIG. 14A-J shows that S. aureus PSMα leads to disruption of epithelial barrier homeostasis. Human keratinocytes (NHEKs) were stimulated with S. aureus (SA) sterile-filtered supernatant from wild type (WT), PSMα (ΔPSMα) or PSMβ (ΔPSMβ) knockout strains for 24 h and (A) trypsin activity and (B) KLK6 mRNA compared to the housekeeping gene GAPDH were analyzed (n=4). (C) PSM synthetic peptides were added to NHEKs for up to 24 h to analyze changes in trypsin activity. (D,E) Transcript analysis by RNA-Seq of genes that changed ≥2 fold after PSMα3 treatment was assessed followed by gene ontology (GO) analysis. 8 week old male C57BL/6 mice (n=6) were treated for 72 h with SA WT, SA ΔPSMα, or a SA 10 secreted protease knockout strain (Δproteases) (le⁷ CFU). (F,G) Murine skin representative pictures (dashed lines indicate treatment area) and changes to epidermal thickness after treatment (scale=200 μm). (H-K) Changes in murine back skin with WT or mutant SA strains in transepidermal water loss (TEWL) and SA CFU/cm2 were assessed as well. All error bars are represented at standard error of the mean (SEM) and One-way ANOVAs were used to determine statistical significance indicated by: p<0.05*, p<0.01**, p<0.001***, p<0.0001****.

FIG. 15A-G shows Staphylococcus epidermidis agr type I auto-inducing peptide characterization and deficiency in AD skin. (A,B) S. epidermidis agr types I-III supernatant inhibition of S. aureus (SA) USA300 LAC agr type I activity after 24 h (n=4) and representation of known structure of S. epidermidis agr type I autoinducing peptide (AIP). (C) Staphylococcus epidermidis (S. epi) agr type I strain RP62A wild-type (WT) or autoinducing peptide knockout (ΔAIP) effect on SA agr activity after 24h. (D) SA sterile-filtered supernatant growth with or without S. epi WT or ΔAIP supernatant was applied to NHEKs for an additionally 24h followed by measurement of NHEK trypsin activity (n=4). (E) Consensus of S. epidermidis agr types I-III genomes found on AD skin. (F,G) Ratio of S. epidermidis agr type I to SA relative abundance on flare regions of 8 individual AD subjects from ‘least severe’ to ‘most severe’ AD score based upon objective SCORAD and overall combined data of all subjects based upon AD severity. All error bars are represented at standard error of the mean (SEM) and One-way ANOVAs (A,C,D) and a (nonparametric) unpaired Mann-Whitney test (F) were used to determine statistical significance indicated by: p<0.05*, p<0.01**, p<0.001***, p<0.0001****.

FIG. 16A-F shows multiple clinically isolated Coagulase-negative Staphylococci inhibit S. aureus agr activity. (A) Sterile-filtered supernatants of clinically isolated Coagulase-negative Staphylococci (CoNS) were added to S. aureus (SA) USA300 LAC agr type I P3-YFP reporter strain for 24 h followed by analysis for SA agr activity (n=3). (B,C) S. hominis C5 strain genome was further sequenced and analyzed at the agrD gene for the auto-inducing peptide (AIP) sequence. Biochemical analysis of S. hominis C5 supernatant tested the ability of a <3 kDa size exclusion centrifugation filtration, 80% ammonium sulfate precipitate, and pH11 1 h treated supernatant to effect SA agr activity as well. (D-F) SA grown in presence of S. hominis C5 supernatant for 24 h was sterile filtered and added to human keratinocytes (NHEKs) for 24h followed by analysis of trypsin activity, KLK6 mRNA expression compared to the housekeeping gene GAPDH, and IL-6 protein levels. All error bars are represented at standard error of the mean (SEM) and One-way ANOVAs were used to determine statistical significance indicated by: p<0.05*, p<0.01**, p<0.001***, p<0.0001****.

FIG. 17A-H shows AD clinical CoNS isolate inhibits SA induced murine skin barrier damage. S. aureus (SA) USA300 LAC agr type I pAmi P3-Lux reporter strain (le⁷ CFU) with or without live S. hominis C5 (le⁸ CFU) was applied to 8 week female C57BL/6 mice for 48 h (n=5). (A,B) SA agr activity was assessed on murine back skin by changes in luminescence. (C) Representative images of murine skin after 48 h SA treatment (dashed boxes indicate treatment area). (D-H) SA CFU/cm² was determined and murine skin barrier damage and inflammation was assessed by analyzing changes in 116 mRNA expression, transepidermal water loss (TEWL), trypsin activity, and Klk6 mRNA expression normalized to the housekeeping gene Gapdh. All error bars are represented at standard error of the mean (SEM) and One-way ANOVAs were used to determine statistical significance indicated by: p<0.05*, p<0.01**, p<0.001***, p<0.0001****.

FIG. 18A-H shows that S. aureus PSMα changes essential barrier genes and cytokine expression in human keratinocytes. (A-D) Human keratinocytes treated with synthetic PSMα3 were assessed for changes in trypsin activity and KLK6 transcript expression normalized to the housekeeping gene GAPDH in both a dose and time dependent manner. (E) GO-term analysis of genes down-regulated 2 fold from the control in human keratinocytes treated with PSMα3 for 24h. (F-H) Changes in human keratinocyte cytokine protein expression of IL-6, TNF-α, or IL-1α treated with SA WT, SA Δpsmα, or SA Δpsmβ supernatant for 24h. All error bars are represented at standard error of the mean (SEM) and One-way ANOVAs were used to determine statistical significance indicated by: p<0.05*, p<0.01**, p<0.001***, p<0.0001****.

FIG. 19A-H shows that S. aureus PSMα and proteases are responsible for barrier damage and induction of inflammation on murine skin. S. aureus (SA) (le⁷ CFU) wild type (WT), PSMα knockout (Δpsmα), and protease null (Δproteases) strains were applied to male murine back skin for 72 h (n=6) and changes in (A,E) trypsin activity, (B,F) Klk6, (C,G) 116, and (D,H) IL17a/f mRNA expression normalized to the housekeeping gene Gapdh were measured. All error bars are represented at standard error of the mean (SEM) and One-way ANOVAs were used to determine statistical significance indicated by: p<0.05*, p<0.01**, p<0.001***, p<0.0001****.

FIG. 20A-C shows that CoNS strains do not effect SA growth. Coagulase-negative Staphylococci (CoNS) supernatant affect on SA agr type I P3-YFP reporter strain growth as assessed by OD600 nm (n=3-4) including (A) CoNS clinical isolates, (B) S. epidermidis (S. epi) agr type I-III, and (C) S. epidermidis (S. epi) wild type (WT) or auto-inducing peptide knockout (ΔAIP) supernatant added to SA agr type I reporter strain for 24h. All error bars are represented at standard error of the mean (SEM).

FIG. 21A-B shows that S. hominis C5 inhibits SA agr type I-III but not type IV. S. hominis C5 supernatant added to SA agr types I-IV P3-YFP reporter strains for 24 h (n=3). (A) SA reporter strain agr type I-IV activity and (B) measurement of growth by OD600 nm when cultured in presence of S. hominis C5 supernatant. All error bars are represented at standard error of the mean (SEM) and One-way ANOVAs were used to determine statistical significance indicated by: p<0.05*, p<0.01**, p<0.001***, p<0.0001****.

FIG. 22A-F shows S. hominis C5 supernatant inhibits SA induced skin barrier damage. S. aureus (SA) (le⁷ CFU) with or without 10× concentrated <3 kDa S. hominis C5 supernatant was applied to female murine back skin for 48 h (n=3). (A-B) Representative images of murine back (dashed lines indicate treatment area) and SA CFU/cm² recovered from murine skin after SA treatment. (C-F) SA induced skin barrier damage markers including 116, transepidermal water loss (TEWL), trypsin activity, and Klk6 mRNA expression compared to the housekeeping gene Gapdh. All error bars are represented at standard error of the mean (SEM) and One-way ANOVAs were used to determine statistical significance indicated by: p<0.05*, p<0.01**, p<0.001***, p<0.0001****.

### DETAILED DESCRIPTION

As used herein and in the appended claims, the singular forms “a,” “an,” and “the” include plural referents unless the context clearly dictates otherwise. Thus, for example, reference to “an agent” includes a plurality of such agents and reference to “the microorganism” includes reference to one or more microorganisms and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Any methods and reagents similar or equivalent to those described herein can be used in the practice of the disclosed methods and compositions.

Atopic dermatitis (AD) is among the most common immune disorders, and causes a serious burden to patient quality of life and finances as well as posing a serious risk of comorbidities. Defects in skin barrier function are an important characteristic of AD. Eczematous skin lesions of patients with AD have increased levels of Th2 cytokines such as IL4 and IL13. Th2 cytokines promote decreased function of the skin barrier by inhibiting expression of filaggrin. These cytokines also suppress expression of human antimicrobial peptides such as cathelicidin and b-defensin-2, a defect in AD that may lead to dysbiosis of the skin bacterial community and enhanced colonization by S. aureus. Therapy targeting IL4 receptor alpha result in significant improvement in disease. The strong association between Th2 cytokine activity, barrier function, antimicrobial activity, and disease outcome supports efforts to define a causal link between these essential epidermal functions.

The skin barrier of patients with AD may be compromised by increased proteolytic activity as they have been found to display increased kallikrein (KLK) expression. KLKs are a family of 15 serine proteases of which several are found predominately in the upper granular and stratum corneal layers of the epidermis. In Netherton syndrome, increased serine protease activity is observed due to decreased activity of the serine protease inhibitor Kazal-type 5. The resulting increase in enzymatic activity leads to increased desquamation, altered antimicrobial peptide and filaggrin (FLG) processing, and protease-activated receptor 2 activation and inflammation. Increased protease activity may also play an important role in the communication of the microbiome with the skin immune system, and has recently been shown to directly influence epidermal cytokine production and inflammation by enhancing penetration of bacteria through the epidermis.

Dysbiosis of the skin's microbiome and the colonization of the skin by Staphylococcus aureus is associated with the exacerbations of atopic dermatitis (AD). The present disclosure demonstrates S. aureus has the ability to induce expression of specific KLKs from keratinocytes and increase overall proteolytic activity in the skin. This illustrates a system by which bacteria on the skin communicate with the host and suggests a previously unknown but likely important mechanism for how S. aureus colonization can increase disease severity in patients with AD.

S. aureus can secrete multiple proteases onto the skin that alter skin barrier integrity. The serine protease V8 and serine-like protease exfoliative toxins have been shown to cleave corneodesmosome adhesion proteins including DSG-1 leading to increased desquamation. Aureolysin, an MMP, is known to cleave and inactivate LL-37, an important antimicrobial peptide on the skin. However, these direct proteolytic actions of S. aureus products require high levels of the enzyme and bacteria, and are more consistent with events that occur during infection with this organism.

Increased digestion of barrier proteins was observed after keratinocytes were activated by S. aureus. FLG is known to be cleaved from the larger Pro-FLG (400 kDa) into a monomeric form (37 kDa) that plays an important role in forming the physical barrier of the stratum corneum with keratin. It has been shown that accelerated Pro-FLG cleavage could be linked to increased desquamation of the skin (Hewett et al., 2005). Interestingly, increased cleavage of Pro-FLG was observed in human keratinocytes treated with S. aureus supernatant. Pro-FLG cleavage was partially blocked when KLK6 or KLK13 was silenced, indicating that S. aureus may decrease skin barrier integrity in a KLK-dependent manner through cleavage of Pro-FLG.

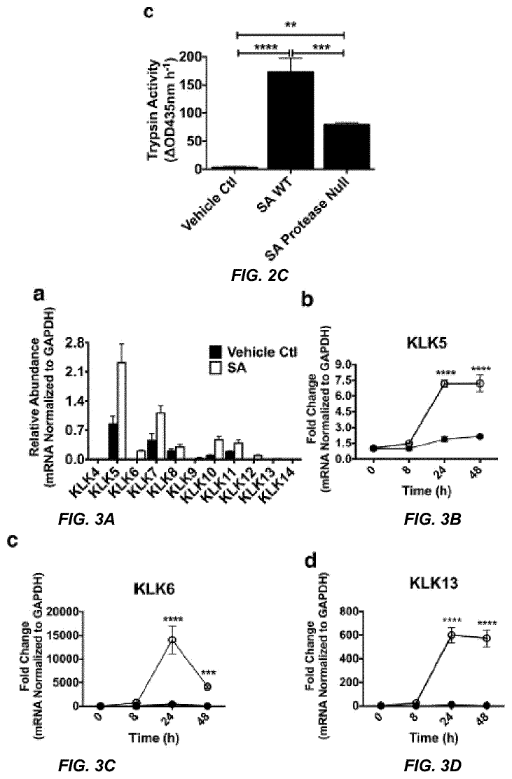

DSG-1 is an important corneodesmosome adhesion protein that when cleaved leads to increased desquamation. Full-length DSG-1 (160 kDa) in keratinocytes is readily cleaved by KLK activity stimulated by S. aureus. It has been reported that KLK5, 6, 7, and 14 can cleave DSG-1, whereas KLK13 could not. This showed that upregulated KLK6 and KLK14 can lead to enhanced cleavage of full-length DSG-1 while providing contrary evidence to the notion that KLK13 is not involved in DSG-1 cleavage. Thus, S. aureus can cause KLKs to alter FLG cleavage, but also increase DSG-1 cleavage as another way to decrease the epidermal skin barrier integrity. Specific siRNA knockdown suggested that the increased expression of KLKs was responsible, at least in part, for the increased serine protease activity stimulated by S. aureus. FIG. 2C demonstrates that secreted proteases from S. aureus contribute to the induction of increased trypsin activity in keratinocytes. Because bacteria including S. aureus can penetrate the skin surface and elicit strong dermal immune responses (Nakatsuji et al., 2013, 2016; Zhang et al., 2015), it is possible that these bacteria may also influence protease activity of dermal cells. These observations also relate to Rosacea or Netherton syndrome.

The disclosure demonstrates that soluble factor(s) produced by S. aureus have a potent and previously unsuspected capacity to alter endogenous protease activity produced by the keratinocyte. This occurred at a dilution of S. aureus products from which the activity of the bacterial proteases was undetectable. Thus, S. aureus can promote the epidermis to increase expression of endogenous proteolytic activity, thus drastically altering the balance of total epidermal proteolytic activity.

Different strains of S. aureus (Newman, USA300, 113, and SANGER252) and S. epidermidis (ATCC12228 and ATCC1457) had different effects on human keratinocyte protease activity. S. aureus strains including Newman and USA300 increased trypsin activity, whereas other strains of S. aureus and S. epidermidis increased elastase or MMP activity. Thus, bacteria could alter epidermal protease activity depending on both the species and strain of bacteria. It is possible that other bacterial species and strains of S. aureus could further uniquely influence the enzymatic balance of human skin. Interestingly, preliminary data have found that purified toll-like receptor ligands do not induce trypsin activity or KLK expression in keratinocytes.

Protease activity is highly upregulated in multiple skin diseases leading to a damaged skin barrier. This is associated with a worsened disease state in almost all cases. The disclosure demonstrates, in one aspect, that commensal microbes and their bacterial products are useful to prevent increased protease activity in the skin. In particular, the disclosure demonstrates that coagulase negative Staphylococci can prevent Staphylococcus aureus induced serine protease activity in the skin by inhibiting the agr quorum sensing system. Staphylococcus aureus, a pathogenic bacteria strain can induce serine protease activity in the skin. Increased protease activity disrupts the skin barrier and leads to worsened disease states including Netherton syndrome and atopic dermatitis. The disclosure demonstrates that this increased serine protease activity can be prevented through use of commensal, or good, skin bacteria and factors derived therefrom.

The disclosure presents an unexpected response of keratinocytes to S. aureus. Because of the increased DSG-1 and FLG cleavage, S. aureus produces one or more factors that decrease the integrity of the skin barrier in a KLK-dependent manner.

The disclosure demonstrates that S. aureus not only secretes proteases but also can specifically activate keratinocytes to increase expression of endogenous proteases. The disclosure demonstrates that phenol-soluble modulin alpha (PSMα) is secreted by S. aureus and triggers auto-digestion of the epidermis. For example, three members of the KLK family appear to play a role in this increased enzymatic activity.

The disclosure also identifies commensal bacteria, genes and polypeptides that inhibit the accessory gene regulator (agr) quorum sensing system of S. aureus and turns off PSMα thereby inhibiting protease activity. Thus, the disclosure provides targets for modulating atopic dermatitis as well as agents and probiotic preparations to modulate atopic dermatitis and protease activity on the skin.

The disclosure demonstrates that coagulase-negative Staphylococci (CoNS) species that normally reside on skin such as S. epidermidis and S. hominis protect against this biological activity of S. aureus by producing auto-inducing peptides (AIP) that inhibit the accessory gene regulatory (agr) quorum sensing system of S. aureus and turn off PSMα secretion.

Virtually all S. aureus toxins are under the control of the virulence accessory gene regulator (agr). The agr system triggers changes in gene expression at a particular cell density by a process called quorum sensing. In addition to toxins, agr is known to upregulate a wide variety of virulence determinants, such as exoenzymes (proteases, lipases, nucleases), and downregulate expression of surface binding proteins. This adaptation is believed to control production of certain virulence determinants of an infection, when they are needed (e.g., binding proteins at the beginning, when cell density is low and adhesion to host tissue is important, and toxins and degradative exoenzymes when the infection is established and nutrients need to be acquired from host tissues.

Multiple clinical isolates of different CoNS species inhibited protease activation and prevented epithelial damage both in vitro and in vivo without changing the abundance of S. aureus (e.g., inhibited the biological activity of protease/agr activity, without changing S. aureus density). Moreover, the disclosure shows that patients with active AD showed a decrease in relative abundance of these beneficial microbes (e.g., CoNS) compared to S. aureus, thus overcoming inhibition of quorum sensing and enabling barrier disruption by S. aureus. Taken together, the disclosure shows how members of the normal human skin microbiome maintain immune homeostasis by contributing as a community to the control of S. aureus toxin production.

The disclosure has also identified polynucleotide sequences, polypeptide sequences and fragments thereof that provide for products that inhibit agr quorum sensing activity. These polynucleotide and polypeptide can be used to provide therapeutics and recombinant non-pathogenic or attenuated skin bacteria for use in topical formulations to treat S. aureus infections and/or atopic dermatitis.

For example, the disclosure provides for auto-inducing peptides (AIPs) that downregulate agr activity. Polynucleotides encoding the AIPs are also provided herein.

The disclosure provides a link between increased S. aureus colonization and increased serine protease activity in AD skin and provides new targets and therapies including, but not limited to, fermentation extracts to either up regulate protease activity (e.g., fermentation extracts from S. aureus) or fermentation extracts from commensal bacteria that down regulate protease activity in the skin (e.g., containing one or more AIPs of the disclosure). Moreover, the disclosure provides for (i) topical formulations comprising such extracts or purified AIPs peptides, (ii) topical formulations comprising commensal probiotic bacteria (e.g., non-pathogenic or attenuated bacterial that have been transformed with an AIP coding sequence, or purified commensal bacterial preparations in a topical formulation). Further therapeutic targets can be antibodies to KLKs, and/or DSG-1 and/or FLG therapy (e.g., increased expression or delivery of these factors to AD subjects).

In one embodiment, an AIP polypeptide of the disclosure has the consensus sequence of X₁X₂X₃X₄CX₅X₆X₇X₈ (SEQ ID NO:10), wherein X1 is S, K, V, G or T; X2 is Y, Q, A, or I; X3 is N, S, T, or D; X4 is V, P, M, or T; X5 is G, S, A, N, or T; X6 is G, N, T, or L; X7 is Y or F; and X8 is F, L, or Y, wherein amino acids 5-9 of SEQ ID NO:10 form a thiolactone ring. Exemplary peptide sequence that fall within the consensus sequence of SEQ ID NO:10 include SYNVCGGYF (SEQ ID NO:4), KYNPCSNYL (SEQ ID NO:11), SYSPCATYF (SEQ ID NO:12), SQTVCSGYF (SEQ ID NO:13), GANPCALYY (SEQ ID NO:14), TINTCGGYF (SEQ ID NO:15), VQDMCNGYF (SEQ ID NO:16), and GYSPCTNFF (SEQ ID NO:17). In a further embodiment, the polypeptide generates a structure of Formula I or IA. In another embodiment, the polypeptide can comprise a combination of D- or L-amino acids. In any of the foregoing embodiments, the polypeptide inhibits S. aureus protease activity, agr activity or keratinocyte protease activity.

The disclosure provides a compound of Formula I

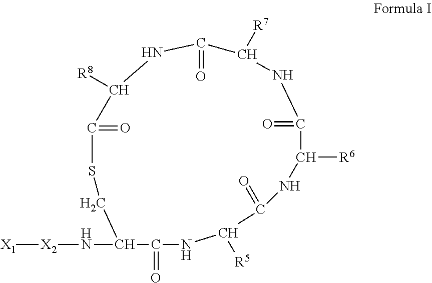

wherein X₁ is from 1-6 amino acids; X2 is an amino acid selected from valine (V), proline (P), methionine (M) and threonine (T); wherein R⁵ is selected from the group consisting of

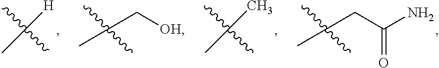

and

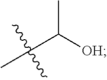

wherein R⁶ is selected from the group consisting of

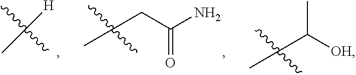

and

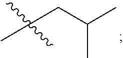

wherein R⁷ is selected from the group consisting of

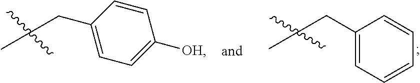 [SMILES: *Cc1ccc(O)cc1 ; "and" ; *Cc1ccccc1 ; ";"]

and wherein R⁸ is selected from the group consisting of

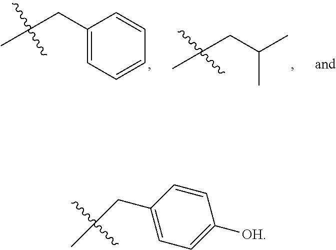

In one embodiment, the disclosure provides a compound of Formula IA:

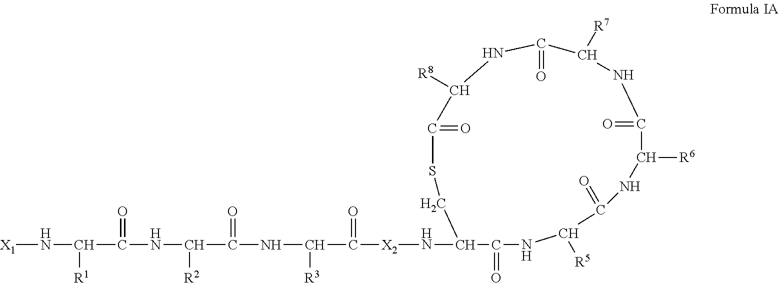

wherein X₁ is from 1-6 amino acids; X2 is an amino acid selected from valine (V), proline (P), methionine (M) and threonine (T); wherein R¹ is selected from the group consisting of

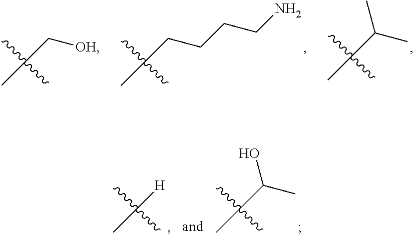

wherein R² is selected from the group consisting of

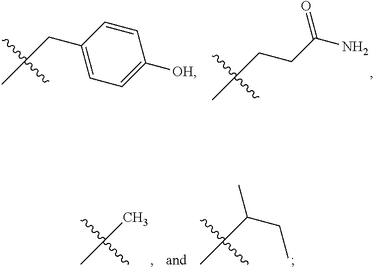

wherein R³ is selected from the group consisting of

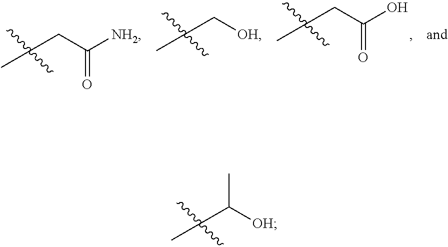

wherein R⁵ is selected from the group consisting of

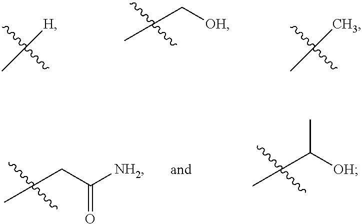

wherein R⁶ is selected from the group consisting of

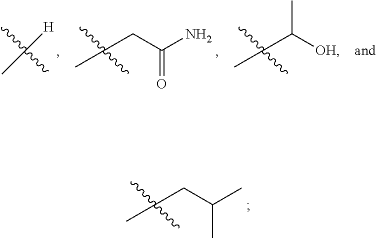

wherein R⁷ is selected from the group consisting of

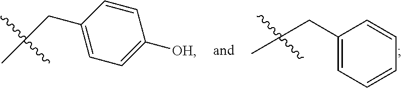 [SMILES: *Cc1ccc(O)cc1 ; *Cc1ccccc1]

and wherein R⁸ is selected from the group consisting of

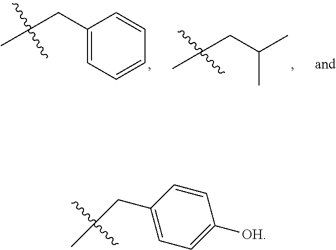

The disclosure provides a purified polypeptide (e.g., an AIP peptide) comprising a sequence that is at least 98% identical to SEQ ID NO:4 and which inhibits (i) protease production and/or protease activity of keratinocytes, (ii) inhibits IL-6 production and/or activity of keratinocytes, (iii) inhibits production of phenol soluble modulin alpha 3 from Staphylococcus aureus (S. aureus) and/or (iv) inhibits agr production and/or activity by S. aureus. In another embodiment, the disclosure provides for a compound of Formula IB:

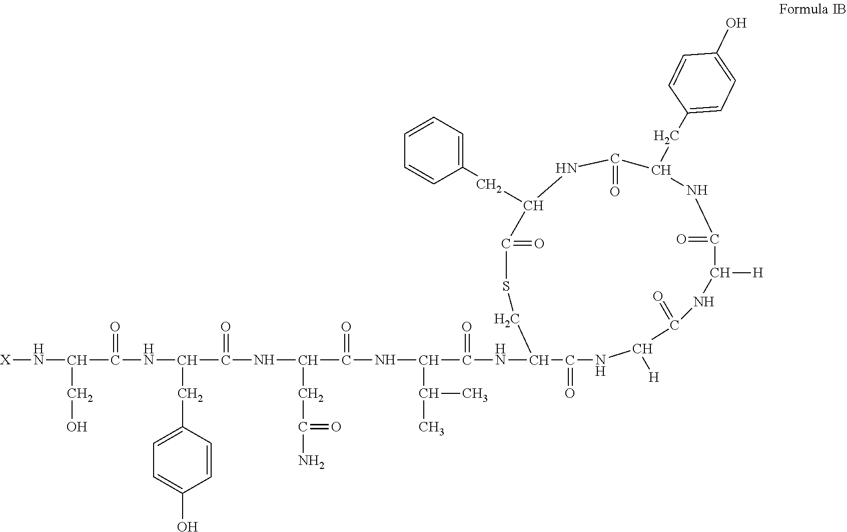

In still a further embodiment, the disclosure provides a purified polypeptide comprising or consisting of SEQ ID NO:4, 11, 12, 13, 14, 15, 16, or 17. In a further embodiment, the polypeptide forms a structure of formula I, IA or IB.

In one embodiment, an AIP peptide of the disclosure can comprise one or more D-amino acids.

The disclosure provides a topical formulation comprising an AIP peptide having a consensus sequence of SEQ ID NO:10 or a peptide of SEQ ID NO:4, 11, 12, 13, 14, 15, 16, or 17 or compound of Formula I, IA or IB.

“Substantially identical” means that an amino acid sequence is largely, but not entirely, the same, but retains a functional activity of the sequence to which it is related. The percent of identity to polypeptide sequence or polynucleotides sequences share is based upon the alignment of the sequence. It is common in the art to use various programs to perform alignment and to determine identity. In general two polypeptides or domains are “substantially identical” if their sequences are at least 85%, 90%, 95%, 98% or 99% identical, or if there are conservative variations in the sequence. A computer program, such as the BLAST program (Altschul et al., 1990) can be used to compare sequence identity.

The disclosure also provides a polynucleotide (i.e., an “AIP polynucleotide”) encoding an AIP polypeptide of the disclosure. For example, the disclosure provides a polynucleotide encoding SEQ ID NO:2 or 4. In one embodiment, the polynucleotide hybridizes under stringent conditions to a polynucleotide consisting of SEQ ID NO:3 and encodes a polypeptide of SEQ ID NO:4. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995). “Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Polynucleotide sequences encoding SEQ ID NO:11, 12, 13, 14, 15, 16, and 17 can be deduced using codon charts.

An AIP polynucleotide can be cloned into various vectors for use in the disclosure. For example, an AIP polynucleotide can be cloned into an expression vector or plasmid for use in transformation and/or expression in a recombinant host cell. Vectors for use in bacterial transformations are known. The four major types of vectors are plasmids, viral vectors, cosmids, and artificial chromosomes. Common to all engineered vectors are an origin of replication, a multicloning site, and a selectable marker. Any of these are suitable for use herein. An AIP polyucleotide can be inserted into a clone, vector, shuttle, plasmid, BAC, or can also be integrated into the bacterial genome. If a plasmid is used, the copy number of the plasmid can be between 5-500 copy numbers per cell. Exemplary plasmids and expression vectors include but are not limited to: p252, p256, p353-2 (Leer et al. 1992), p8014-2, pA1, pACYC, pAJ01, pA1-derived (Vujcic & Topisirovic 1993), pall, pAM-beta-1,2,3,5,8 (simon and chopin 1988), pAR1411, pBG10, pBK, pBM02, pBR322, pBR328, pBS-slpGFP, pC194 (McKenzie et al. 1986, 1987; Horinouchi & Weisblum 1982b), PC194/PUB110, pC30il, pC30il (Skaugen 1989), pCD034-1, pCD034-2, pCD256, pC12000, pC1305, pC1528, pCIS3, pCL2.1, pCT1138, pD125, pE194, pE194/PLS1, pEGFP-C1, pEH, pF8801, pFG2, pFK-series, pGK-series, pGK12, pGK13, pIA, pIAV1,5,6,7,9, pIL.CatT, pIL252/3, pIL253, pIL7, pISA (low for E. coli), pJW563, pKRV3, pLAB1000 (Josson et al. 1990), pLB4 (Bates & Gilbert 1989, pLBS, pLE16, pLEB124, pLEB590, pLEB591, pLEB600, pLEB604, pLEP24Mcop, pLJ1 (Takiguchi et al. 1989), pLKS, pLTK2, pWCFS101 and pMD5057 (Bates & Gilbert, 1989; Skaugen, 1989; Leer et al., 1992; Vujcic & Topisirovic, 1993; Eguchi et al., 2000; Kaneko et al., 2000; Danielsen, 2002; Daming et al., 2003; de las Rivas et al., 2004; van Kranenburg et al., 2005), pLP1/18/30, pLP18, pLP317, pLP317cop, pLP3537, pLP3537xyl, pLP402, pLP825, pLP825 and pLPE323, pLP82H, pLPC37, pLPE23M, pLPE323, pLPE350, pLPI (Bouia et al. 1989), pLS1, pLS1 and pE194 (Lacks et al. 1986; Horinouchi & Weisblum 1982a), plul631, pLUL631 from L. reuteri carrying an erythromycin-resistance gene, pM3, pM4, pMD5057, pMG36e, pND324, pNZ-series, pPSC series, pSH71 (de vos, 1987), pSIP-series, pSK11L, pSL2, PSN2, pSN2 (Khan & Novick 1982), pT181 (Koepsel et al. 1987), (Khan & Novick 1983), pT181, pC194 and pE194 are not functional in B. subtilis (Gruss et al. 1987), pT181, pE194/pLS1, pC194/pUB110 and pSN2 (Khan, 2005), pTL, pTRK family, pTRT family, pTUAT35, pUBII0 and pC194 (McKenzie et al. 1986, 1987; Horinouchi & Weisblum 1982b), pUCL22, pULP8/9, pVS40, pWC1, pWCFS101, pWV02, pWV04, pWV05, RepA, system BetL.

In one embodiment, the disclosure provides a topical composition comprising an AIP polypeptide or peptide of the disclosure. For example, in one embodiment, the topical composition comprises a purified polypeptide (e.g., an AIP peptide) comprising a consensus sequence of SEQ ID NO:10, or a sequence that is at least 98% identical to any of SEQ ID NO:4, 11, 12, 13, 14, 15, 16, or 17 and which inhibits (i) protease production and/or protease activity of keratinocytes, (ii) inhibits IL-6 production and/or activity of keratinocytes, (iii) inhibits production of phenol soluble modulin alpha 3 from Staphylococcus aureus (S. aureus) and/or (iv) inhibits agr production and/or activity by S. aureus. In another embodiment, the topical composition comprises a compound of Formula I, IA, or IB (as defined above).

In another embodiment, the topical composition can comprise a non-pathogenic microorganism (including attenuated microorganism that have been engineered to reduce or eliminate pathogenic activity), wherein the microorganism has been engineered to expression an AIP polypeptide. The microorganism can be engineered to contain a vector and/or AIP polynucleotide. In one embodiment, the microorganism produces a compound of Formula I, IA and/or IB.

In one embodiment, the compositions and methods herein use non-pathogenic bacteria that have been engineered to produce a compound of Formula I, IA and/or IB, by transforming the bacteria with an AIP polynucleotide of the disclosure. In one embodiment, the bacteria in the population are non-pathogenic and non-invasive microorganisms, and can be in certain embodiments a gram-positive food grade bacterial strain. In another embodiment, the populations of transformed bacteria are prepared from a bacterium that occurs naturally in the skin microbiome.

In certain embodiments, bacteria forming the population of bacteria in the composition, and that are transformed to express a compound of Formula I, IA, and/or IB, can be a collection of the same bacteria or a mixture of different bacteria, at different phylogenetic levels. Bacteria resident on the skin of healthy humans include bacterial species typically resident on the face of humans, such as Actinobacteria, including bacterial in the genus Corynebacterium and in the genus Propionibacterium. In other embodiments, bacteria resident on the skin of healthy human subjects include bacterial species typically resident on skin other than the face, including for example bacteria in the genus bacteroidetes and proteobacteria. Other bacteria in the skin microbiome include those listed herein below.

In one embodiment, the bacteria are from the genus Propionibacterium, including but not limited to, Propionibacterium acidifaciens, Propionibacterium acidipropionici, Propionibacterium acidipropionici strain 4900, Propionibacterium acnes, Propionibacterium australiense, Propionibacterium avidum, Propionibacterium cyclohexanicum, Propionibacterium freudenreichii subsp. Freudenreichii, P. freudenreichii ssp. freudenreichii strain 20271, Propionibacterium freudenreichii subsp. Shermanii, P. freudenreichii ssp. shermanii strain 4902, P. freudenreichii ssp. shermanii strain 4902, Propionibacterium granulosum, Propionibacterium innocuum, P. jensenii strain 20278, Propionibacterium lymphophilum, Propionibacterium microaerophilum, Propionibacterium propionicum, Propionibacterium thoenii, and P. thoenii strain 20277. In one embodiment, the bacteria is not Propionibacterium acnes. In one embodiment, the bacteria are from the genus Corynebacterium, including but not limited to, C. accolens, C. afermentan, C. amycolatum, C. argentoratense, C. aquaticum, C. auris, C. bovis, C. diphtheria, C. equi (now Rhodococcus equi), C. flavescens, C. glucuronolyticum, C. glutamicum, C. granulosum, C. haemolyticum, C. halofytica, C. jeikeium (group JK), C. macginleyi, C. matruchotii, C. minutissimum, C. parvum (Propionibacterium acnes), C. propinquum, C. pseudodiphtheriticum (C. hofmannii), C. pseudotuberculosis, (C. ovis), C. pyogenes, C. urealyticum (group D2), C. renale, C. spec, C. striatum, C. tenuis, C. ulcerans, C. urealyticum, and C. xerosis. Bacterial with lipophilic and nonlipophilic groups are contemplated, and the nonlipophilic bacteria may include fermentative corynebacteria and nonfermentative corynebacteria. In one embodiment, the bacteria is not C. diphtheria, C. amicolatum, C. striatum, C. jeikeium, C. urealyticum, C. xerosis, C. pseudotuberculosis, C. tenuis, C. striatum, or C. minutissimum, as these may be pathogenic. In one embodiment, the bacteria are from the suborder Micrococcineae, including but not limited to the GRAS bacteria species Arthrobacter arilaitensis, Arthrobacter bergerei, Arthrobacter globiformis, Arthrobacter nicotianae, Kocuria rhizophila, Kocuria varians, Micrococcus luteus, Micrococcus lylae, Microbacterium gubbeenense, Brevibacterium aurantiacum, Brevibacterium casei, Brevibacterium linens, Brachybacterium alimentarium, and Brachybacterium tyrofermentans. In another embodiment, the bacteria are from the genus Staphylococcus, including but not limited to, Staphylococcus agnetis, S. arlettae, S. auricularis, S. capitis, S. caprae, S. carnosus, Staphylococcus caseolyticus, S. chromogenes, S. cohnii, S. condiment, S. delphini, S. devriesei, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. microti, S. muscae, S. nepalensis, S. pasteuri, S. pettenkoferi, S. piscifermentans, S. pseudintermedius, S. pseudolugdunensis, S. pulvereri, S. rostra, S. saccharolyticus, S. saprophyticus, S. schleiferi, S. sciuri, S. simiae, S. simulans, S. stepanovicii, S. succinus, S. vitulinus, S. warneri, and S. xylosus. In one embodiment, the bacteria is not S. aureus or S. epidermidis. In another embodiment, the bacteria are from the genus Streptococcus, including but not limited to, Streptococcus acidominimus, Streptococcus adjacens, Streptococcus agalactiae, Streptococcus alactolyticus, Streptococcus anginosus, Streptococcus australis, Streptococcus bovis, Streptococcus caballi, Streptococcus canis, Streptococcus caprinus, Streptococcus castoreus, Streptococcus cecorum, Streptococcus constellatus, Streptococcus constellatus subsp. Constellatus, Streptococcus constellatus subsp. Pharyngis, Streptococcus cremoris, Streptococcus criceti, Streptococcus cristatus, Streptococcus danieliae, Streptococcus defectives, Streptococcus dentapri, Streptococcus dentirousetti, Streptococcus didelphis, Streptococcus difficilis, Streptococcus durans, Streptococcus dysgalactiae, Streptococcus dysgalactiae subsp. Dysgalactiae, Streptococcus dysgalactiae subsp. Equisimilis, Streptococcus entericus, Streptococcus equi, Streptococcus equi subsp. Equi, Streptococcus equi subsp. Ruminatorum, Streptococcus equi subsp. Zooepidemicus, Streptococcus equines, Streptococcus faecalis, Streptococcus faecium, Streptococcus ferus, Streptococcus gallinaceus, Streptococcus gallolyticus, Streptococcus gallolyticus subsp. Gallolyticus, Streptococcus gallolyticus subsp. Macedonicus, Streptococcus gallolyticus subsp. Pasteurianus, Streptococcus garvieae, Streptococcus gordonii, Streptococcus halichoeri, Streptococcus hansenii, Streptococcus henryi, Streptococcus hyointestinalis, Streptococcus hyovaginalis, Streptococcus ictaluri, Streptococcus infantarius, Streptococcus infantarius subsp. Coli, Streptococcus infantarius subsp. Infantarius, Streptococcus infantis, Streptococcus iniae, Streptococcus intermedius, Streptococcus intestinalis, Streptococcus lactarius, Streptococcus lactis, Streptococcus lactis subsp. Cremoris, Streptococcus lactis subsp. Diacetilactis, Streptococcus lactis subsp. Lactis, Streptococcus lutetiensis, Streptococcus macacae, Streptococcus macedonicus, Streptococcus marimammalium, Streptococcus massiliensis, Streptococcus merionis, Streptococcus minor, Streptococcus mitis, Streptococcus morbillorum, Streptococcus mutans, Streptococcus oligofermentans, Streptococcus oralis, Streptococcus orisratti, Streptococcus ovis, Streptococcus parasanguinis, Streptococcus parauberis, Streptococcus parvulus, Streptococcus pasteurianus, Streptococcus peroris, Streptococcus phocae, Streptococcus plantarum, Streptococcus pleomorphus, Streptococcus pluranimalium, Streptococcus plurextorum, Streptococcus pneumonia, Streptococcus porci, Streptococcus porcinus, Streptococcus porcorum, Streptococcus pseudopneumoniae, Streptococcus pseudoporcinus, Streptococcus pyogenes, Streptococcus raffinolactis, Streptococcus ratti, Streptococcus rupicaprae, Streptococcus saccharolyticus, Streptococcus salivarius, Streptococcus salivarius subsp. Salivarius, Streptococcus salivarius subsp. Thermophilus, Streptococcus sanguinis, Streptococcus shiloi, Streptococcus sinensis, Streptococcus sobrinus, Streptococcus suis, Streptococcus thermophilus, Streptococcus thoraltensis, Streptococcus tigurinus, Streptococcus troglodytae, Streptococcus troglodytidis, Streptococcus uberis, Streptococcus urinalis, Streptococcus vestibularis, and Streptococcus waius. In another embodiment, the bacteria are from the genus Lactobacillus, including but not limited to, Lactococcus garvieae, Lactococcus lactis, Lactococcus lactis subsp. cremoris, Lactococcus lactis subsp. hordniae, Lactococcus lactis, Lactococcus lactis subsp. Lactis, Lactococcus piscium, Lactococcus plantarum, Lactococcus raffinolactis, Lactobacillus acetotolerans, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarius, Lactobacillus aviarius subsp. araffinosus, Lactobacillus aviarius subsp. aviarius, Lactobacillus bavaricus, Lactobacillus bifermentans, Lactobacillus brevis, Lactobacillus buchneri, Lactobacillus bulgaricus, Lactobacillus carnis, Lactobacillus casei, Lactobacillus casei subsp. alactosus, Lactobacillus casei subsp. casei, Lactobacillus casei subsp. pseudoplantarum, Lactobacillus casei subsp. rhamnosus, Lactobacillus casei subsp. tolerans, Lactobacillus catenaformis, Lactobacillus cellobiosus, Lactobacillus collinoides, Lactobacillus confusus, Lactobacillus coryniformis, Lactobacillus coryniformis subsp. coryniformis, Lactobacillus coryniformis subsp. torquens, Lactobacillus crispatus, Lactobacillus curvatus, Lactobacillus curvatus subsp. curvatus, Lactobacillus curvatus subsp. melibiosus, Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus divergens, Lactobacillus farciminis, Lactobacillus fermentum, Lactobacillus formicalis, Lactobacillus fructivorans, Lactobacillus fructosus, Lactobacillus gallinarum, Lactobacillus gasseri, Lactobacillus graminis, Lactobacillus halotolerans, Lactobacillus hamsteri, Lactobacillus helveticus, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefuranofaciens, Lactobacillus kefirgranum, Lactobacillus kunkeei, Lactobacillus lactis, Lactobacillus leichmannii, Lactobacillus lindneri, Lactobacillus malefermentans, Lactobacillus mali, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus paracasei subsp. paracasei, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus reuteri, Lactobacillus rhamnosus, Lactobacillus rhamnosus strain 5/E5a, Lactobacillus rimae, Lactobacillus rogosae, Lactobacillus ruminis, Lactobacillus sakei, Lactobacillus sakei subsp. camosus, Lactobacillus sakei subsp. sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, Lactobacillus yamanashiensis subsp. mali, Lactobacillus yamanashiensis subsp. Yamanashiensis and Lactobacillus zeae. In another embodiment, the bacteria are from the genus Lactococcus, including but not limited to, Lactococcus Schleifer, Lactococcus chungangensis, Lactococcus fujiensis, Lactococcus garvieae, Lactococcus lactis, Lactococcus lactis subsp. Cremoris, Lactococcus lactis subsp. Hordniae, Lactococcus lactis subsp. Lactis, Lactococcus lactis subsp. Tructae, Lactococcus piscium, Lactococcus plantarum, and Lactococcus raffinolacti.

In yet another embodiment, the disclosure provides a probiotic composition for topical delivery comprising a CoNS commensal skin bacteria of the disclosure. In one embodiment, the CoNS bacteria comprises a bacterial that produces an AIP polypeptide and/or a compound of Formula I. In a further embodiment, the topical composition contains only a single species of microorganisms that produce an AIP polypeptide or compound of Formula I. In still another embodiment, the commensal skin bacteria of the disclosure comprise a microorganism selected from the group consisting of S. epidermidis A11, S. hominis A9, S. hominis C4, S. hominis C5, and S. warneri G2. In still another embodiment, a topical probiotic composition of the disclosure can comprise or consist of a commensal skin bacteria selected from the group consisting of S. epidermidis A11, S. hominis A9, S. hominis C4, S. hominis C5, S. warneri G2, and any combination thereof.

A commensal bacterial of the disclosure can be isolated from human skin and identified using methods described herein. For example, the disclosure provides a method of obtaining, identifying and culturing a commensal bacteria described herein by swabbing human skin surface using, e.g., a foam tip swab. The swabs were placed in tryptic soy broth. The broth is diluted onto mannitol salt agar plates (MSA) supplemented with 3% egg yolk. Pink colonies without halo representing coagulase-negative Staphylococci (CoNS) strains are collected and grown in tryptic soy broth (TSB) prior to addition of sterile-filtered supernatant at 25% by volume to a S. aureus agr type I YFP reporter strain grown in fresh TSB (for measurement of S. aureus agr activity inhibition after a 24 h incubation). Agr activity of S. aureus reporter strain is measured using a fluorometer. Strains with strong inhibition of S. aureus agr activity are further characterized by gDNA isolation and sequencing. gDNA is isolated using any number of commercially available kits (e.g., DNeasy UltraClean Microbial Kit, Qiagen). The gDNA can be sequenced using various sequence platforms (e.g., MiSeq; Illumin Inc., San Diego, Calif.) for two cycles, which can generated 2×250 bp paired-end reads. Adapters are removed using cutadapt (see, e.g., world-wide-web at cutadapt.readthedocs.io/en/stable/). Low-quality sequences can be removed using Trim Galore (see, e.g., world-wide-web at bioinformatics.babraham.ac.uk/projects/trim_galore/) with default parameters. Sequences mapping to the human genome are removed from the quality-trimmed dataset using the Bowtie2 program (ver. 2.28)(1) with parameters (−D 20 −R 3 −N 1 −L 20—very-sensitive-local) and the human reference genome hg19. The filtered reads are de novo assembled using SPAdes (version 3.8.0) with k-mer length ranging from 33-127. The genome is annotated with rapid annotation of microbial genomes using subsystems technology (RASY) with default parameters. Amino acid sequences from annotated CDS (coding DNA sequences) are aligned to bacterial agr proteins obtained from Uniprot database. Agr genes from the assembled genome are identified following three criteria: (i) sequence identity >60%, (ii) e-value <e100; and (iii) the agr locus organization, an operon of four genes, agrBDCA. Microorganism with a sequence that is at least 60%, 70%, 80%, 90%, 95%, 98%, 99% or 100% identical to the sequence of SEQ ID NO:1 or 3 are useful in the methods and compositions of the disclosure.

As used herein, the term “probiotic composition” or “topical probiotic composition” or “probiotic skin composition” includes a composition, which include a probiotic commensal skin bacteria, a probiotic commensal skin bacteria fermentation extract, an attenuated or engineered microorganism that expresses an AIP polypeptide and an agent that (i) inhibits protease activity or (ii) promotes protease activity, and a pharmaceutical carrier that maintain the viability of the commensal skin bacteria.

As used herein, the term “Topical” can include administration to the skin externally, as well as shallow injection (e.g., intradermally and intralesionally) such that a topical probiotic composition comes in direct contact with skin.

As used herein, the term “Fermentation Extract” means a product of fermenting a probiotic commensal skin bacteria in a culture and under appropriate fermentation conditions. For example, culturing S. aureus can produce PSMα3 useful for increasing skin barrier permeability. An extract from S. aureus contains PSMα3 that can be applied to the skin to improve permeability, induce skin remodeling or to promote skin barrier permeability for drug delivery. Similarly, a fermentation extract of a CoNS bacteria that produces an AIP of the disclosure can be cultured and the extract from such culture used to inhibit S. aureus associated pathology (e.g., protease activity, dermatitis etc.).

As used herein, the term “Probiotic Commensal Skin Bacteria” includes a microorganism of the skin microbiome. The probiotic commensal skin bacteria can include a composition of bacterial that promotes protease activity (a “Protease promoting probiotic commensal skin bacteria”). Protease promoting probiotic commensal skin bacteria is typically a bacteria of the skin that produces phenol soluble module alpha 3 (PSMα3). A protease promoting probiotic commensal skin bacterial composition (or fermentation extract thereof) are useful, e.g., for promoting skin remodeling, wound repair, aging, sun damage, pigment abnormalities and scarring. In one embodiment, a protease promoting probiotic commensal skin bacteria comprises one or more bacteria the have serine protease activity and/or induce serine protease activity of the skin. For example, a protease promoting probiotic commensal skin bacteria can include an S. aureus strain that produces phenol soluble module alpha 3 (PSMα3).

In another embodiment, the probiotic commensal skin bacteria can include a composition of bacteria that inhibits protease activity (a “Protease inhibiting probiotic commensal skin bacteria”). A protease inhibiting probiotic commensal skin bacterial composition are useful for treating disease such as rosacea, atopic dermatitis and Netherton syndrome. In one embodiment, a protease inhibiting probiotic commensal skin bacteria comprises one or more bacteria that inhibit serine protease activity of other bacteria of the skin and/or inhibit serine protease activity of the skin. For example, a protease inhibiting probiotic commensal skin bacteria can include a coagulase negative Staphylococci sp. In one embodiment, the coagulase negative strain is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus hominis, Staphylococcus jettensis, Staphylococcus petrasii, and Staphylococcus lugdunensis. In one embodiment, the protease inhibiting commensal skin bacteria is selected from the group consisting an S. epidermidis strain, S. hominis strain, S. warneri strain and any combination thereof. In specific embodiments, the S. epidermidis strain is S. epidermidis 14990 and/or S. epidermidis A11. In another embodiment, the S. hominis strain is S. hominis C4, S. hominis C5, and/or S. hominis A9. In still another specific embodiment, the S. warneri strain is S. warneri G2. In one embodiment, the CoNS bacteria comprises a bacteria that produces an AIP polypeptide and/or a compound of Formula I. In a further embodiment, the topical composition contains only a single species of microorganisms that produce an AIP polypeptide or compound of Formula I. In still another embodiment, the commensal skin bacteria of the disclosure comprise a microorganism selected from the group consisting of S. epidermidis A11, S. hominis A9, S. hominis C4, S. hominis C5, and S. warneri G2. In still another embodiment, the a topical probiotic composition of the disclosure can comprise or consist of a commensal skin bacteria selected from the group consisting of S. epidermidis A11, S. hominis A9, S. hominis C4, S. hominis C5, S. warneri G2, and any combination of the foregoing.

The term “contacting” refers to exposing the skin to a topical probiotic composition such that the probiotic skin composition can modulate protease activity (e.g., serine protease activity) on the skin.

The terms “inhibiting” or “inhibiting effective amount” refers to the amount of probiotic skin composition consisting of one or more probiotic microorganism and/or fermented medium or extract and/or fermentation by-products and/or synthetic molecules that is sufficient to cause, for example, inhibition of protease activity (e.g., serine protease activity) on the skin or in a skin culture. The term “inhibiting” also includes preventing or ameliorating a sign or symptoms of a disorder (e.g., a rash, sore, and the like).

The term “therapeutically effective amount” as used herein for treatment of a subject afflicted with a disease or disorder means an amount of a probiotic skin composition or extract thereof sufficient to ameliorate a sign or symptom of the disease or disorder. For example, a therapeutically effective amount can be measured as the amount sufficient to decrease a subject's symptoms of dermatitis or rash by measuring the frequency of severity of skin sores. Typically, the subject is treated with an amount to reduce a symptom of a disease or disorder by at least 50%, 90% or 100%. Generally, the optimal dosage will depend upon the disorder and factors such as the weight of the subject, the type of bacteria, the sex of the subject, and degree of symptoms. Nonetheless, suitable dosages can readily be determined by one skilled in the art.

The term “purified” and “substantially purified” as used herein refers to cultures, or co-cultures of microorganisms or of biological agent (e.g. fermentation media and extracts, fractionated fermentation media, fermentation by-products, an AIP peptide, polypeptide, gene, polynucleotide, compound of formula I etc.) that is substantially free of other cells or components found in the natural environment with which an in vivo-produced agent would naturally be associated. In some embodiments, a co-culture probiotic can comprise a plurality of commensal skin bacteria.

The disclosure provides whole cell preparations comprising a substantially homogeneous preparation of S. epidermidis, S. hominis and/or S. warneri. Such a preparation can be used in the preparation of compositions for the treatment of inflammation and microbial infections. Whole cell preparation can comprise S. epidermidis, S. hominis and/or S. warneri or may comprise non-pathogenic (e.g., attenuated microbe) vector as described below. The disclosure also provides fractions derived from such whole cells comprising agents the reduce protease activity in the skin resulting from S. aureus activity.

The ability of a first bacterial composition to inhibit the protease activity of a second bacterial composition can be determined by contacting measuring the protease activity of the second bacterial composition before and after contacting the second composition with the first composition. Contacting of an organism with a topical probiotic composition of the disclosure can occur in vitro, for example, by adding the topical probiotic composition to a bacterial culture to test for protease inhibitory activity of the bacteria. Alternatively, contacting can occur in vivo, for example by contacting the topical probiotic composition with a subject afflicted with a skin disease or disorder.

A probiotic commensal skin bacterial preparation can be prepared in any number of ways. Any of a variety of methods known in the art can be used to administer a topical probiotic compositions to a subject. For example, a probiotic skin composition or extract or synthetic preparation of the disclosure may be formulated for topical administration (e.g., as a lotion, cream, spray, gel, or ointment). Such topical formulations are useful in treating or inhibiting microbial, fungal, viral presence or infections or inflammation on the skin. Examples of formulations include topical lotions, creams, soaps, wipes, and the like.

In yet another embodiment, a topical probiotic composition is provided that comprises a plurality of probiotic commensal skin bacteria. When used for the treatment of dermatitis or other skin diseases or disorders associated with increased protease (e.g., serine protease) activity, the composition comprises one or more bacteria that inhibit protease activity on the skin. In such instances, the probiotic commensal skin bacteria is a coagulase negative Staphylococcus sp. In one embodiment, the probiotic commensal skin bacterial is selected from the group consisting of S. epidermidis strain, S. hominis strain, S. warneri strain and any combination thereof. Where increased protease activity is desired (e.g., for the treatment of wounds, skin remodeling, etc.), the probiotic commensal bacterial composition contains bacteria that have increase protease activity or that stimulate skin protease activity (e.g., serine protease activity). In this embodiment, an exemplary commensal bacterial composition will comprise an S. aureus bacteria or a virulence-attenuated S. aureus that produces PSMα3.

In another embodiment, the topical probiotic composition comprises a probiotic commensal skin bacteria fermentation extract that promotes protease activity on the skin. In various aspects, the bacteria from which the extract is produced comprises Staphylococcus aureus.

In yet another embodiment, a topical probiotic composition is provided consisting essentially of S. aureus fermentation extract alone or in combination with an S. aureus. In accordance with a further aspect, the topical probiotic composition above can be formulated as a lotion, shake lotion, cream, ointment, gel, foam, powder, solid, paste or tincture.

In another embodiment, the topical probiotic composition comprises a probiotic commensal skin bacteria fermentation extract. In various aspects, the bacteria from which the extract is produced comprise a coagulase negative Staphylococcus species. In one embodiment, the Staphylococcus species is selected from the group consisting of S. epidermidis strain, S. hominis strain, S. warneri strain and any combination thereof that produce an AIP that inhibits agr quorum sensing system and/or protease production in the skin or microbiome of the skin. In one embodiment, the AIP comprises a consensus sequence of SEQ ID NO:10 or a sequence that is at least 98% identical to SEQ ID NO:4, 11, 12, 13, 14, 15, 16, or 17 having agr quorum modulating activity and/or a compound of Formula I, IA or IB.

In yet another embodiment, a topical probiotic composition is provided consisting essentially of a coagulase negative Staphylococcus sp. fermentation extract or an S. epidermidis fermentation extract alone or in combination with a coagulase negative Staphylococcus sp. or an S. epidermidis. In another embodiment, the composition comprises one or more of the deposited microorganism strains described herein (e.g., S. epidermidis A11, S. hominis A9, S. hominis C5 and/or S. warneri G2).

In accordance with a further embodiment, the topical probiotic composition above can be formulated as a lotion, shake lotion, cream, ointment, gel, foam, powder, solid, paste or tincture.

In another embodiment, a fermentation extract is provided which can be obtained by fermenting a bacteria selected from the group consisting of S. epidermidis strain, S. hominis strain, S. warneri strain and any combination thereof under fermentation conditions. In various aspects, such fermentation extracts can be used for inhibiting serine protease activity on the skin. In another embodiment, the fermentation extract is obtained from any one or more of the deposited microorganism strains described herein (e.g., S. epidermidis A11, S. hominis A9, S. hominis C5 and/or S. warneri G2). In accordance with a further embodiment, the fermentation extract can be formulated as a lotion, shake lotion, cream, ointment, gel, foam, powder, solid, paste or tincture.

In another embodiment, a bandage or dressing is provided comprising the topical probiotic compositions described above, a probiotic commensal skin bacteria fermentation extract described above, a probiotic commensal skin bacteria described above, and any combination thereof. In various aspects, a bandage or dressing is provided the major constituents of which includes a matrix and a probiotic commensal skin bacteria that inhibits protease activity on the skin. In various aspects, a bandage or dressing is provided the major constituents of which includes a matrix and a probiotic commensal skin bacteria fermentation extract that inhibits protease activity on the skin.

In another embodiment, a bandage or dressing is provided comprising the topical probiotic compositions described above, a probiotic commensal skin bacteria fermentation extract described above, a probiotic commensal skin bacteria described above, and any combination thereof. In various aspects, a bandage or dressing is provided the major constituents of which includes a matrix and a probiotic commensal skin bacteria that promotes protease activity on the skin. In various aspects, a bandage or dressing is provided the major constituents of which includes a matrix and a probiotic commensal skin bacteria fermentation extract that promotes protease activity on the skin.

The disclosure also provides a method for treating a disease or disorder of the skin associated with protease (e.g., serine protease activity). Example of such disease or disorder include Netherton syndrome, atopic dermatitis, contact dermatitis, eczema, psoriasis, acne, epidermal hyperkeratosis, acanthosis, epidermal inflammation, dermal inflammation and pruritus. In one embodiment, the presence of the disease or disorder is first determined by measuring protease activity of a sample (e.g., of the skin or a culture of bacteria from the skin) from a subject suspected of having the disease or disorder. If the sample, shows higher than normal protease activity (e.g., serine protease activity) then the subject is treated with a protease inhibitory commensal bacterial preparation by contacting the skin of the subject with the preparation. In another embodiment, a culture from the subject having high protease activity and comprising bacteria is contacted with a preparation in vitro to determine the susceptibility of the culture to the preparation and its effect on protease inhibition.

A protease inhibitory commensal bacteria preparation or fermentation extract can be combined with one or more known serine protease inhibitors. There are a number of commercially and clinically relevant serine protease inhibitors that can be used in the methods and compositions of the disclosure. For example, serine protease inhibitors such as those disclosed in, for example, U.S. Pat. Nos. 5,786,328, 5,770,568, or U.S. Pat. No. 5,464,820, the disclosures of which are incorporated herein by reference. Exemplary serine protease inhibitory agents include antibodies that bind to and inhibit a serine protease polypeptide or functional fragment thereof, enzymes that degrade a serine protease polypeptide to inactive peptides, substrate analogs, and the like. A serine protease expression inhibitor includes, for example, antisense molecules, ribozymes and small molecule agents (e.g., vitamin D antagonists) that reduce the transcription or translation of a serine protease polynucleotide (e.g., DNA or RNA). One embodiment of the disclosure is directed to substrate analogs of tissue kallikrein. These substrate analogs comprise a peptide with an amino acid sequence corresponding to positions 388 to 390 of tissue kallikrein. Peptides may be made synthetically, genetically by recombinant engineering techniques, such as by cloning and expressing of a nucleic acid sequence, or purified from natural sources such as a bacterial, fungal or cellular extracts. The structure, chemical, physicochemical, nomenclature and analytical aspects of amino acids are described in Chemistry of the Amino Acids (J. P. Greenstein and M. Winitz editors, John Wiley & Sons, New York, N.Y., 1961, reprinted 1984), which is hereby specifically incorporated by reference. The peptides are comprised of modified and/or unmodified amino acids which include the naturally occurring amino acids, the non-naturally occurring (non-coding) amino acids, synthetically made amino acids, and combinations thereof. The naturally occurring amino acids include glycine (Gly), the amino acids with alkyl side chains such as alanine (Ala), valine (Val), leucine (Leu), isoleucine (Ile), and proline (Pro), the aromatic amino acids phenylalanine (Phe), tyrosine (Tyr) and tryptophan (Trp), the amino acid alcohols serine (Ser) and threonine (Thr), the acidic amino acids aspartic acid (Asp) and glutamic acid (Glu), the amides of Asp and Glu, asparagine (Asn) and glutamine (Gln), the sulfur-containing amino acids cysteine (Cys) and methionine (Met), and the basic amino acids histidine (His), lysine (Lys), and arginine (Arg). The non-naturally occurring amino acids include, for example, ornithine (Orn), norleucine (Nle), citralline (Cit), homo-citralline (hCit), desmosine (Des), and isodesmosine (Ide). Modified amino acids include derivatives and analogs of naturally and non-naturally occurring, and synthetically produced amino acids. Such amino acid forms have been chemically modified such as, for example, by halogenation of one or more active sites with chlorine (Cl), bromine (Br), fluorine (F), or iodine (I), alkylation with a carbon containing group such as a methyl (Me), ethyl (Et), butyl (Bu), amino (NH2 or NH3), amidino (Am), acetomidomethyl (Acm), or phenyl (Ph) group, or by the addition of a phosphorous (P), nitrogen (N), oxygen (O) or sulfur (S) containing group. Modifications may also be made by, for example, hydration, oxidation, hydrogenation, esterification, or cyclization of another amino acid or peptide, or of a precursor chemical. Examples include the amino acid hydroxamates and decarboxylases, the dansyl amino acids, the polyamino acids, and amino acid derivatives. Specific examples include gamma amino butyric acid (GABA), hydroxyproline (Hyp), aminoadipic acid (Aad) which may be modified at the 2 or 3 position, o-aminobutyric acid (Aab or Abu), selenocysteine (SeCys2), tert-butylglycine (Bug or tert-BuGly), the N-carbamyl amino acids, the amino acid methyl esters, amino-propionic acid (or β-alanine; 13-Ala), adamentylglycine (Adg), aminocaproic acid (Acp), N-ethylasparagine (Et-Asn), allo-hydroxylysine (aHyl), allo-isoleucine (aIle), phenylglycine (Phg), pyridylalanine (Pal), thienylalanine (Thi), α-Δ-aminobutyric acid (Kbu), α-β-diaminopropionic acid (Kpr), 1- or 2-naptithylalanine (1Nal or 2Nal), orthofluorophenylalanine (Phe(o-F)), N-methylglycine (MeGly), N-methyl-isoleucine (Melle), N-methyl-valine (MeVal), 2-amino-heptanoic acid (Ahe), 2- or 3-amino-isobutyric acid (Aib), 2-amino-pimellic acid (Dbu), 2-2′-diaminopimellic acid (Dpm), 2,3-diaminopropionic acid (Dpr), and N-ethylglycine (EtGly). Chemically produced non-coded amino acids include, for example, phenylglycine (Ph-Gly), cyclohexylalanine (Cha), cyclohexylglycine (Chg), and 4-amino phenylalanine (Phe(4NH2) or Aph). Modified amino acids may also be chemical structures which are not amino acids at all, but are actually classified as another chemical form such as an alkyl amine, a saccharide, a nucleic acid, a lipid, a fatty acid or another acid. Any of the modified or unmodified amino acids which comprise the peptide may be in the D- or L-conformations or comprise one, two or more tautomeric or resonance forms.

A pharmaceutical composition comprising a probiotic skin composition disclosed herein comprising a commensal bacteria (e.g., S. epidermidis A11, S. hominis A9, S. hominis C4, S. hominis C5 and/or S. warneri G2), an engineered form thereof (e.g., attenuated or genetically modified), or an attenuated microorganism comprising an AIP peptide coding sequence may be formulated in any dosage form that is suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation comprising a probiotic disclosed herein may also comprise liposomes, micelles, microspheres, nanosystems, and mixtures thereof.

In one embodiment, a bandage or dressing is provided comprising a probiotic skin composition disclosed herein comprising a commensal bacteria (e.g., S. epidermidis A11, S. hominis A9, S. hominis C4, S. hominis C5 and/or S. warneri G2), an engineered form thereof (e.g., attenuated or genetically modified), or an attenuated microorganism comprising an AIP peptide coding sequence described herein. In various aspects, a bandage or dressing is provided the major constituents of which includes a matrix and a probiotic skin composition comprising a commensal bacteria (e.g., S. epidermidis A11, S. hominis A9, S. hominis C4, S. hominis C5 and/or S. warneri G2), an engineered form thereof (e.g., attenuated or genetically modified), or an attenuated microorganism comprising an AIP peptide coding sequence described above. In various embodiments, a bandage or dressing is provided the major constituents of which includes a matrix and a probiotic commensal skin bacteria or extract. In various aspects, a bandage or dressing is provided the major constituents of which includes a matrix and a probiotic commensal skin bacteria fermentation extract. In various aspects, a bandage or dressing is provided the major constituents of which includes a matrix and glycerol. In one embodiment, the bandage or dressing is applied to site of skin damage or injury. In another embodiment, the bandage or dressing is applied to a site of infection.

A “pharmaceutically acceptable carrier” is intended to include solvents, dispersion media, coatings, antibacterial and antifungal agents (as needed so long as they are not detrimental to the probiotic commensal bacteria), isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

Pharmaceutically acceptable carriers and excipients suitable for use in the topical formulations disclosed herein include, but are not limited to, aqueous vehicles, water-miscible vehicles, non-aqueous vehicles, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

A pharmaceutical composition comprising a probiotic may be formulated in the forms of ointments, creams, sprays and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, glycerol and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain material substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

In another embodiment, a pharmaceutical composition comprising a compound of Formula I and/or a commensal probiotic disclosed herein, a derivative or analog thereof, can be formulated either alone or in combination with one or more additional therapeutic agents, including, but not limited to, chemotherapeutics, antibiotics (so long as they don't destroy the probiotic benefits), antifungal-agents, anti-pruritics, analgesics, protease inhibitors and/or antiviral agents.

Topical administration, as used herein, include (intra)dermal, conjunctival, intracorneal, intraocular, ophthalmic, auricular, transdermal, nasal, vaginal, uretheral, respiratory, and rectal administration. Such topical formulations are useful in treating or inhibiting cancers of the eye, skin, and mucous membranes (e.g., mouth, vagina, rectum). Examples of formulations in the market place include topical lotions, creams, soaps, wipes, and the like.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In yet a further embodiment, a composition (e.g., a probiotic composition or a composition comprising a peptide or compound of Formula I) provided herein can be combined with one or more steroidal drugs known in the art, including, but not limited to, aldosterone, beclometasone, betamethasone, deoxycorticosterone acetate, fludrocortisone acetate, hydrocortisone (cortisol), prednisolone, prednisone, methylprenisolone, dexamethasone, and triamcinolone.

In yet a further embodiment, a composition (e.g., a probiotic composition or a composition comprising a peptide or compound of Formula I) provided herein can be combined with one or more anti-fungal agents, including, but not limited to, amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compositions (e.g., a probiotic composition or a composition comprising a peptide or compound of Formula I) provided herein, optionally in combination with another agent as disclosed herein. Such kits optionally comprise a composition disclosed herein with an identifying description or label or instructions relating to its use in the methods described herein.

The following EXAMPLES are provided to further illustrate but not limit the invention.

### EXAMPLES

### Example 1

Culture of primary human keratinocytes. Neonatal NHEKs (ThermoFisher Scientific, Waltham, Mass.) were cultured in EpiLife medium (ThermoFisher Scientific) supplemented with 1× EpiLife defined growth supplement (ThermoFisher Scientific), 60 μM CaCl₂), and 1× antibiotic-antimycotic (PSA; 100 U/ml penicillin, 100 U/ml streptomycin, 250 ng/ml amphotericin B; ThermoFisher Scientific) at 37° C., 5% CO2. For experiments, NHEKs were grown to 70% confluency followed by differentiation in high calcium EpiLife medium (2 mM CaCl₂)) for 48 hours before treatment with bacteria sterile filtered supernatant. Use of these human-derived commercial cell products does not require informed consent. For bacterial supernatant treatments, differentiated NHEKs were treated with sterile filtered bacterial supernatant at 5% by volume to EpiLife medium. NHEKs were only used for experiments between passages 3 and 5.

Bacterial culture. All bacteria were cultured in 3% tryptic soy broth (TSB; Sigma, St. Louis, Mo.) at 37° C. with shaking at 300 r.p.m. S. aureus strains Newman, USA300, 113, SANGER252 and S. epidermidis strains ATCC12228 and ATCC1457 were grown for 24 hours to stationary phase followed by centrifugation (4,000 r.p.m., room temperature [RT], 10 minutes) and sterile filtration (0.22 μm) of supernatants before addition to NHEKs. Briefly, the protease-null strain was cultured for 24 hours in 3% TSB containing 25 μg/ml lincomycin and 5 μg/ml erythromycin followed by subculture in 3% TSB only for an additional 24 hours. For murine live S. aureus colonization assays, 2×10⁶ colony-forming units of bacteria were applied to 8-mm TSB agar disks and allowed to dry for 30 minutes at RT before addition to murine dorsal skin.

Murine bacteria disk model. Female C57BJ/6L mice (8 weeks old) were used for a murine model of bacterial skin colonization. Briefly, to remove dorsal skin hair, mice were shaved and nair was applied for 2e3 minutes followed by removal of hair with alcohol wipes. After 24 hours of recovery, 3×8 mm TSB agar disks were applied to murine dorsal skin with TSB only (vehicle control) or 2e⁶ colony-forming units of S. aureus (USA300) per disk for 12 hours. Tegaderm was applied on top of agar disks to hold in place. Mice were euthanized followed by the collection of 8-mm whole skin punch biopsies for analysis.

In situ zymography. Murine skin sections (10 μm thickness) were rinsed 1× with 1% tween-20 in water for 5 minutes. The sections were treated with 2 μg ml of BODIPY FL casein total protease activity substrate (Thermo-Fisher Scientific) for 4 hours at 37° C. in a humidified chamber for the measurement of total protease activity. The serine protease inhibitor AEBSF (50 mM; Sigma) was applied to sections 30 minutes before the addition of the BODIPY FL casein as well. Slides were rinsed 1× in phosphate buffered saline followed by application of ProLong gold antifade mounting medium without DAPI (ThermoFisher Scientific) and a cover slide. Fluorescent signal was measured using an Olympus BX51 (Tokyo, Japan) fluorescent microscope.

Protease activity assays. The NHEK conditioned medium was added at 50 ml to 96-well black bottom plates (Corning, Corning, N.Y.) followed by the addition of 150 ml of 5 μg ml BODIPY FL casein substrate, 2 μg ml of elastin (elastase-like substrate; ThermoFisher Scientific), or 4 μg ml gelatin (MMP substrate; ThermoFisher Scientific) according to the manufacturer's instructions. Additionally, 200 μM of the peptide Boc-Val-Pro-Arg-AMC (trypsin-like substrate; BACHEM, Bubendorf, Switzerland) was added to the NHEK conditioned medium at 150 μl in 1× digestion buffer (ThermoFisher Scientific). Relative fluorescent intensity was analyzed with a SpectraMAX Gemini EM fluorometer (ThermoFisher Scientific) at RT with readings every 2 hours for 24 hours. BODIPY FL casein plates were read at ex: 485 nm and em: 530 nm. Elastin-like and MMP substrate plates were read at ex: 485 nm and em: 515 nm. Trypsin-like substrate plates were read at ex: 354 nm and em: 435 nm.

Quantitative real-time PCR. RNA was isolated from NHEKs using Purelink RNA isolation columns (ThermoFisher Scientific) according to the manufacturer's instructions. RNA was quantified using a Nanodrop spectrophotometer (ThermoFisher Scientific), and 500 ng of RNA was reverse-transcribed using the iScript cDNA synthesis kit (Bio-Rad, Irvine, Calif.). Quantitative real-time PCR reactions were ran on a CFX96 real-time detection system (Bio-Rad) using gene-specific primers and TaqMan probes (ThermoFisher Scientific).

Immunoblotting. For cell lysis, cold 1× radio-immunoprecipitation assay (RIPA) buffer (Sigma) containing 1× protease inhibitor cocktail (Cell Signaling Technology, Danvers, Mass.) was applied to NHEKs followed by scraping. Cell lysates were incubated for 30 minutes on ice and centrifuged (13,000 r.p.m., 15 minutes, 4° C.) to remove cell debris. Samples were prepared by determining protein concentration with bicinchoninic acid (BCA) assays (Pierce, Rockford, Ill.) followed by the addition of 40 mg of sample to 4× Laemmli sample buffer (Bio-Rad) containing 1% b-mercaptoethanol and heating for 7 minutes at 95° C. Samples were ran on 4-20% tris-glycine precast TGX gels (Bio-Rad), transferred to 0.22-μm polyvinylidene difluoride (PVDF) membranes (Bio-Rad) using a trans-blot turbo transfer system (Bio-Rad), blocked for 1 hour at RT in 1× odyssey blocking solution containing 0.1% tween-20 (LI-COR, Lincoln, Nebr.), and stained overnight at 4° C. with primary antibodies. Odyssey (LI-COR) fluorescent secondary antibodies were applied to membranes for 1 hour at RT on an orbital shaker after 3×PBST (phosphate buffered saline with 0.1% tween-20) washes. additional 3×PBST washes were applied before analysis on an infrared imager (LI-COR). The primary antibodies KLK5 (H-55), KLK6 (H-60), DSG-1 (H-290), FLG (H-300), and a-tubulin (TU-02) from Santa Cruz Biotechnologies (Santa Cruz, Calif.) were used at 1:100 dilutions. KLK13 (ab28569) and KLK14 (ab128957) antibodies from Abcam (Cambridge, UK) were used at 1:1,000 dilutions.

KLK gene silencing. NHEKs were treated for 24 hours with 15 nM or 45 nM of specific KLK silencer select siRNA or a siRNA scrambled (−) control (ThermoFisher Scientific) using RNAiMAX (ThermoFisher Scientific) and OptiMEM medium (ThermoFisher Scientific). NHEKs were differentiated in high calcium medium (2 mM CaCl₂)) for 48 hours followed by a 24-hour treatment with sterile filtered S. aureus (Newman) supernatant before the analysis of NHEK lysates and the conditioned medium.

Statistical analysis. Both one-way analyses of variance and two-way analyses of variance were used for statistical analysis with a P value <0.05 being significant. GraphPad prism version 6.0 (GraphPad, La Jolla, Calif.) was used for statistical analysis of results.

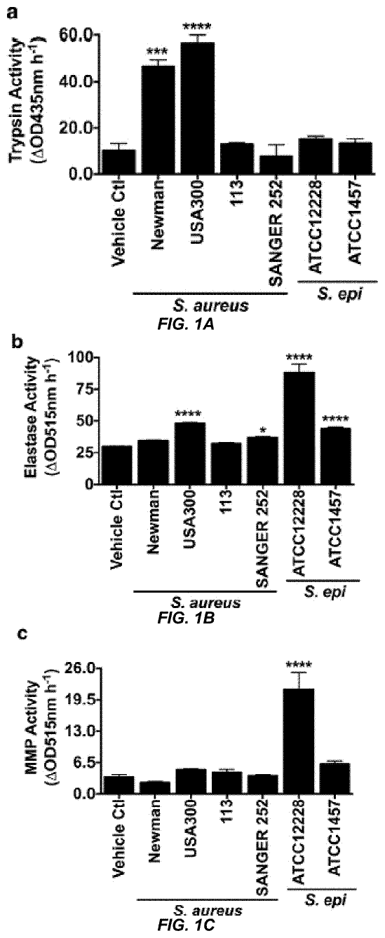

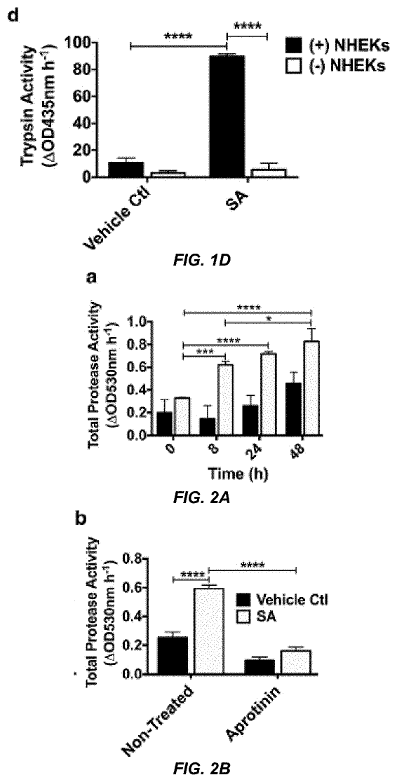

Staphylococci affect the protease activity of human keratinocytes. To evaluate if different strains of bacteria found on human skin can induce protease activity of keratinocytes, primary cultures of normal human epidermal keratinocytes (NHEKs) were treated with sterile filtered culture supernatant from four different laboratory isolates of S. aureus including two methicillin-resistant S. aureus strains (USA300 and SANGER252) and two methicillin-sensitive S. aureus strains. Two S. epidermidis isolates (ATCC12228 and ATCC1457) were also tested. Twenty-four hours after exposure to the sterile bacterial culture supernatants, the keratinocyte culture media was analyzed for protease activity with substrates selective for trypsin-like, elastase-like, or matrix metalloproteinase (MMP) activity. The NHEK conditioned medium contained significantly more trypsin activity after treatment with S. aureus strains Newman and USA300 (FIG. 1a). Both MMP and elastase activity were increased by S. epidermidis strain ATCC12228, whereas the S. aureus strains USA300 and SANGER 252 and the S. epidermidis strain ATCC1457 increased elastase activity to a lesser extent in the NHEK conditioned medium (FIGS. 1b and c). To confirm that the increased protease activity observed in the NHEK conditioned medium was derived from NHEKs and not produced by the bacteria themselves, trypsin activity was analyzed after the addition of S. aureus (Newman) supernatant to culture wells with and without the presence of NHEKs. No enzymatic activity was detected in the absence of NHEKs when the same concentration of diluted supernatant from S. aureus was added to the NHEK media alone (FIG. 1d).

S. aureus increases epidermal serine protease activity. Because of the large increase in trypsin activity induced by certain S. aureus strains (Newman and USA300), and the potential role this activity could have in diseases mediated by S. aureus, experiments were focused on this organism to better understand how the bacteria induce protease activity in NHEKs. To evaluate the kinetics of the protease response to S. aureus, keratinocytes were treated for 0, 8, 24, and 48 hours with sterile filtered culture supernatant from S. aureus (Newman) and then the NHEK conditioned medium was collected for protease analysis. Measurement of total protease activity in the conditioned medium of NHEKs showed a time-dependent increase in total proteolytic activity after exposure to S. aureus supernatant (FIG. 2a). The addition of the serine protease inhibitor aprotinin confirmed that this activity was due to serine proteases (FIG. 2b), and this was consistent with the observation of an increase in trypsin-like activity shown in FIG. 1a. A comparison of S. aureus USA300 LAC wild-type and a protease-null strain demonstrated that both the wildtype and protease-null strains increased trypsin activity in the NHEK conditioned medium, but the protease-null strain had significantly decreased capacity to induce trypsin activity compared with that of the wild-type strain (FIG. 2c). Together, these data confirm that S. aureus can increase endogenous NHEK serine protease activity and that S. aureus proteases and other S. aureus products contribute to the ability of this bacterium to activate keratinocytes.

To further validate the action of S. aureus on epidermal protease activity, live S. aureus (USA300) was applied to the back skin of mice. Skin at the site of application was then biopsied and sectioned for analysis of total proteolytic activity by in situ zymography in the presence or absence of the serine protease inhibitor 4-benzenesulfonyl fluoride (AEBSF). Total epidermal protease activity was qualitatively increased in the epidermis after treatment with S. aureus compared with skin treated with agar disks alone, and the increased activity detected by increased fluorescence was largely eliminated by inhibition of serine protease activity with AEBSF. Background autofluorescence at hair follicles was observed in all sections including the no substrate control. These observations further demonstrate that the presence of S. aureus can increase protease activity in the epidermis.

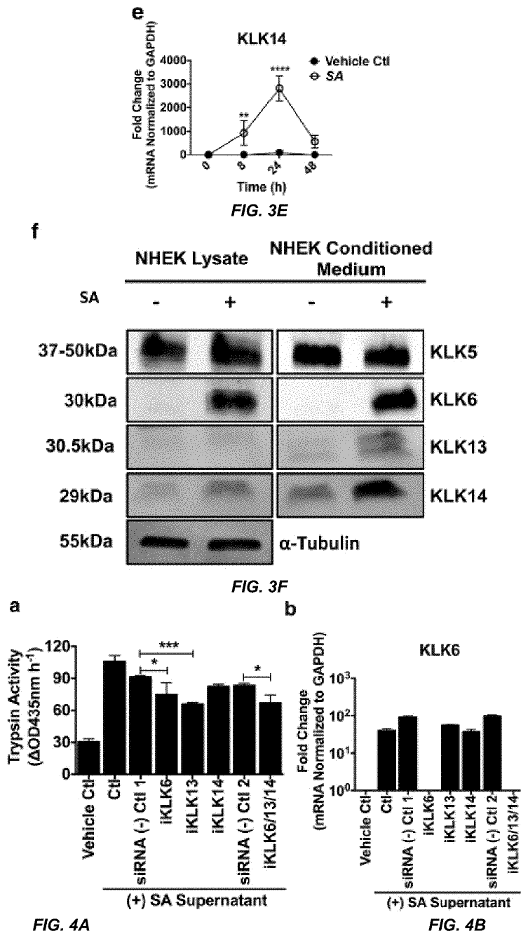

S. aureus increases KLK expression in keratinocytes. KLKs are an abundant serine protease family in the epidermis that have trypsin-like or chymotrypsin-like activity. To determine if S. aureus could change the expression of KLK mRNA in keratinocytes, NHEKs were treated for 24 hours with S. aureus (Newman) supernatant and expression of KLK1-15 was measured by quantitative real-time PCR. KLK5 had the highest relative mRNA abundance, whereas KLK6, 13, and 14 consistently displayed the largest fold increase after exposure to S. aureus (FIG. 3a-e). All other KLKs analyzed showed subtle increases in mRNA expression after exposure to S. aureus except KLK1 that showed decreased expression. mRNA for KLK2, 3, and 15 were not detected.

Both cell lysates and NHEK conditioned medium were then analyzed for changes in KLK protein expression after S. aureus (Newman) supernatant treatment. Immunoblotting for KLK6 and 14 displayed increased expression of these KLK proteins after S. aureus supernatant treatment in both the cell lysate and the conditioned medium, whereas KLK13 was only increased in the conditioned medium. KLK5 had no change in expression after S. aureus supernatant treatment (FIG. 3f).

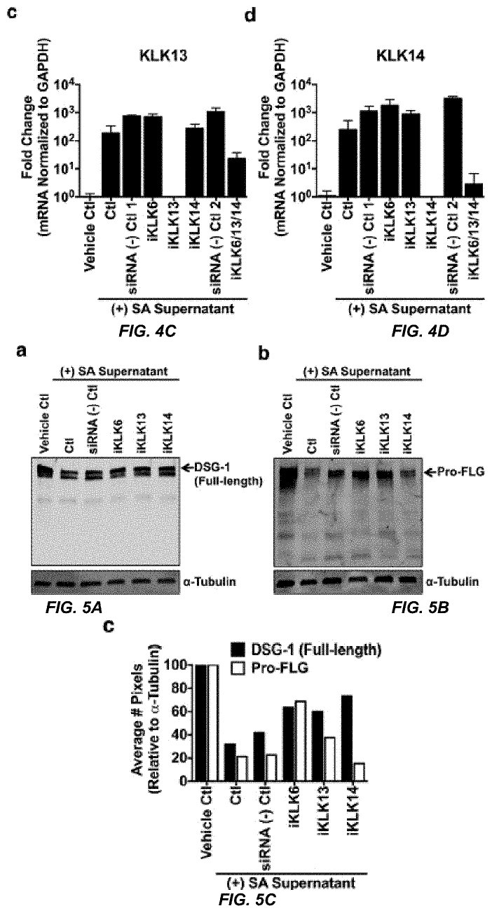

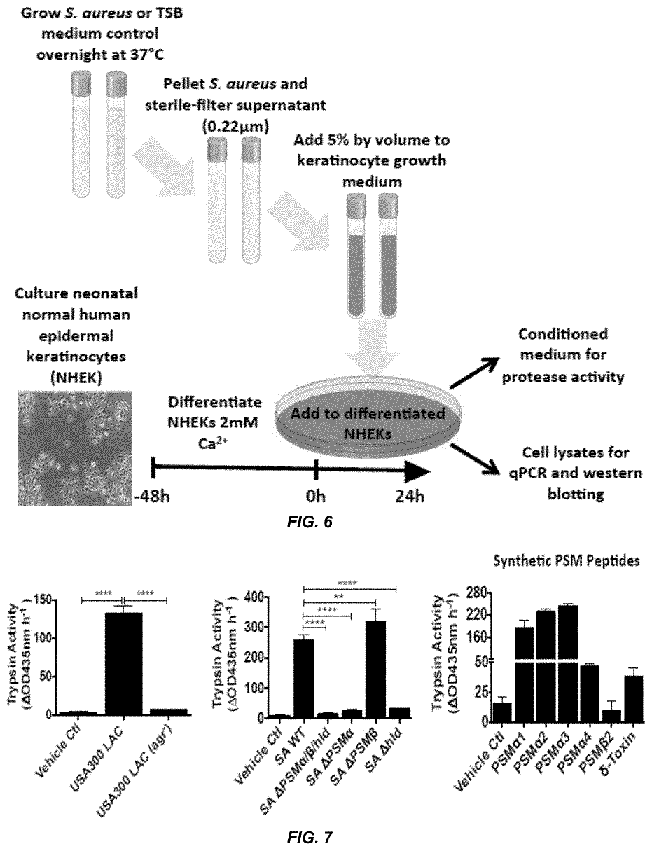

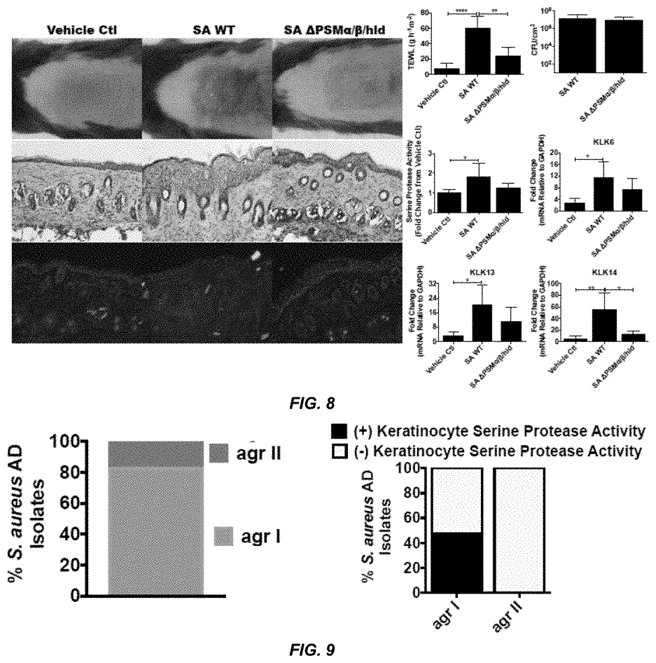

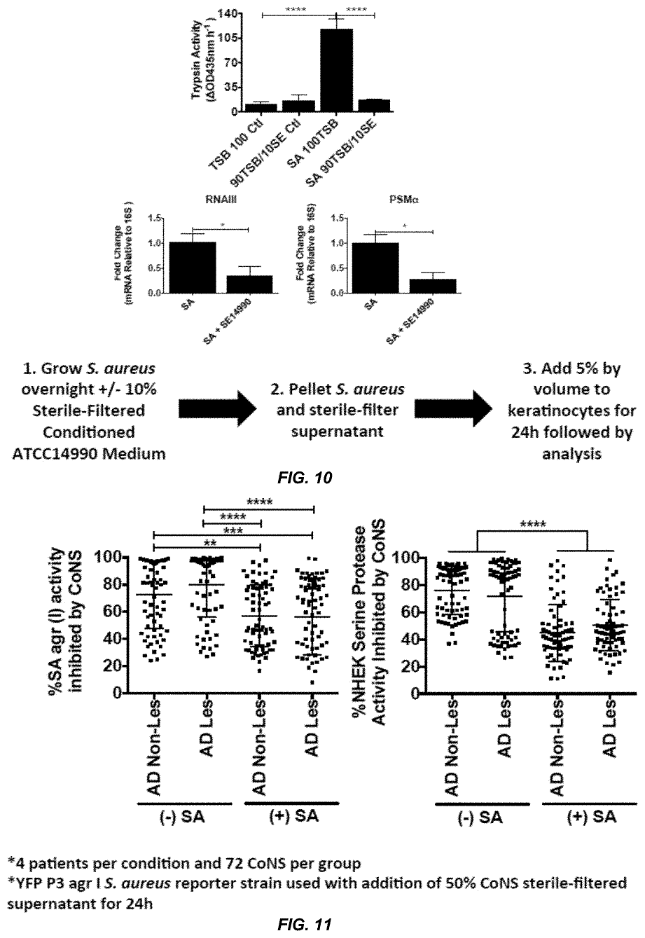

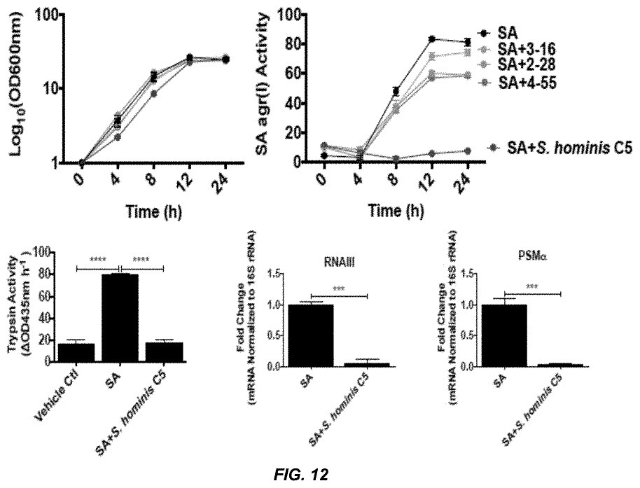

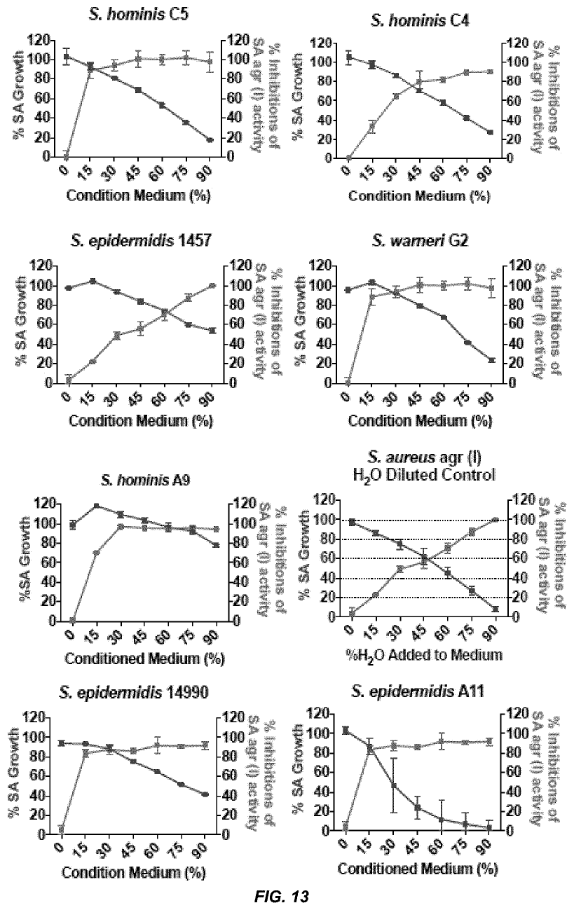

KLK6, 13, and 14 contribute to increased keratinocyte serine protease activity. Because KLK6, 13, and 14 showed the largest increase in expression in NHEKs after S. aureus exposure, experiments were performed to examine if these KLKs were responsible for the observed increased serine protease activity. Small interfering RNA (siRNA) was used to selectively silence their expression. siRNA for KLK6 and KLK13 significantly decreased S. aureus induced trypsin activity, whereas KLK14 decreased trypsin activity to a lesser extent. A triple knockdown of KLK6, 13, and 14 also showed a significant decrease in trypsin activity from the control siRNA although an additive effect was not observed (FIG. 4a). Interestingly, triple knockdown of KLK6, 13, and 14 led to decreased knockdown efficiency of KLK13 and KLK14, which may account for the lack of an additive effect for trypsin activity (FIG. 4b-d).

S. aureus promotes degradation of desmoglein-1 and FLG by induction of KLKs. Desmoglein-1 (DSG-1) and FLG are both important for regulating the epidermal skin barrier integrity. Immunoblotting showed that exposure of NHEKs to S. aureus (Newman) supernatant promoted the cleavage of full length DSG-1 (160 kDa), and that DSG-1 cleavage was blocked by siRNA silencing of KLK6, 13, or 14 (FIG. 5a). S. aureus-mediated cleavage of profilaggrin (Pro-FLG) in NHEKs, indicated by the >250 kDa band on the immunoblot, was also partially blocked by siRNA silencing of KLK6 and KLK13 (FIG. 5b). Densitometry analysis further illustrates the ability of KLK6, 13, and 14 knockdowns to prevent either DSG-1 or Pro-FLG cleavage (FIG. 5c). Overall, these observations demonstrate that the capacity of S. aureus to increase keratinocyte proteolytic activity by induction of KLK6, 13 and 14 can lead to digestion of molecules essential for maintaining a normal epidermal barrier.

### Example 2

Bacterial Preparation. All bacteria used in this study are listed in Table A. All Staphylococci strains (S. aureus, S. epidermidis, S. hominis, S. warneri, S. capitis, and S. lugdunensis) were grown to stationary phase in 3% tryptic soy broth (TSB) for 24 h at 250 RPM in a 37° C. incubator at either 4 mL or 400 μL volumes depending on the assay. Specific strains were grown with antibiotic selection where indicated in Table S1 at the following concentrations: 5 μg/mL Erm, 25 μg/mL Lcm, and 10 μg/mL Cm. For treatment of bacterial supernatant on human keratinocytes or murine skin, 24 h cultured bacteria was pelleted (15 min, 4,000 RPM, RT) followed by filtered-sterilization of the supernatant (0.22 μm). For murine and human keratinocyte experiments with S. hominis C5 and S. epidermidis RP62A strains, bacteria sterile-filtered supernatant was filtered with a 3 kDa size exclusion column (Amicon Ultra-15 centrifugual filter, Millipore) to collect the <3 kDa fraction and further concentrated 10× using a lyophilizer and a re-suspended in molecular grade H₂O prior to treatment. S. hominis C5 supernatant was further biochemically tested with several techniques. Ammonium sulfate precipitation (80%) for 1 h at room temperature followed by centrifugation (30 min, 4,000 RPM, RT) and re-suspension of the precipitate (pellet) in H₂O was used for isolating small peptides. Furthermore, S. hominis C5 supernatant was raised to pH11 with 2M NaOH for 1 h followed by using 2M HCl to return the supernatant pH to approximately the starting pH of 6.5 using pH 1-14 strips prior to addition to the S. aureus agr reporter strain.

TABLE A
Bacterial and Plasmids
Bacteria
Strain Name
S. epidermidis RP62A WT (agr type I)
S. epidermidis RP62A ΔAIP (#47)
S. epidermidis ATCC1457 (agr type II)
S. epidermidis 8247 (agr type III)
S. epidermidis A9
S. epidermidis A11
S. aureus USA300 LAC WT
S. aureus USA300 Δpsmα
S. aureus USA300 Δpsmβ
S. aureus USA300 LAC WT (AH1263)
S. aureus USA300 LAC Δprotease
(AH1919), Erm^{R}. Lcm^{R}
S. aureus USA300 LAC agr type I pAmi
P3-Lux (AK2759), Cm^{R}
S. aureus USA300 LAC agr type I P3-YFP
(AH1677), Cm^{R}
S. aureus 502a agr type II P3-YFP
(AH430), Cm^{R}
S. aureus MW2 agr type III P3-YFP
(AH1747), Cm^{R}
S. aureus MN TG agr type IV P3-YFP
(AH1872), Cm^{R}
S. hominis C4
S. hominis C5
S. hominis A9
S. warneri G25
S. capitis H8
S. lugdunensis E7
DC10B-CC10
Plasmids
Strain Name
pMAD (Amp^{R} in E. coli, Erm^{R} in
Staphylococci)
pMAD:: ΔAIP

Normal Human Keratinocyte Culture. Normal neonatal human epidermal keratinocytes (NHEKs; Thermo Fisher Scientific) were cultured in Epilife medium containing 60 μM CaCl₂ (Thermo Fisher Scientific) supplemented with 1× Epilife Defined Growth Supplement (EDGS; Thermo Fisher Scientific) and 1× antibiotic-antimycotic (PSA; 100 U/mL penicillin, 100 U/mL streptomycin, 250 ng/mL amphotericin B; Thermo Fisher Scientific) at 37° C., 5% CO₂. NHEKs were only used for experiments between passages 3-5. For experiments, NHEKS were grown to 70% confluency followed by differentiation in high calcium EpiLife medium (2 mM CaCl₂) for 48h to simulate the upper layers of the epidermis. For bacterial supernatant treatments, differentiated NHEKs were treated with sterile-filtered bacterial supernatant at 5% by volume to Epilife medium for 24h. Similarly for synthetic PSM treatments, 5-50 μg-mL of peptide were added to the NHEKs for 24 h in DMSO.

S. aureus epicutaneous Mouse Model. Sex and age matched male or female C57BL/6 (Jackson) mice at 8 weeks age were used for all experiments (n=3-6) as specified in the figure legends. All animal experiments were approved by the Institutional Animal Care and Use Committee. Mouse hair was removed by shaving and application of Nair for 2-3 min followed by immediate removal with alcohol wipes. The skin barrier was allowed to recover from hair removal for 48 h prior to application of bacteria. S. aureus (1e7 CFU) in 3% TSB was applied to murine skin for 48-72 h at a 100 μL volume on a 1.5 cm² piece of sterile gauze. Tegaderm was applied on top of gauze to hold in place for duration of the treatment. For S. aureus agr inhibition experiments, live S. hominis C5 (10:1) or 10× concentrated <3 kDa sterile-filtered commensal bacterial supernatant (1:1) was combined with S. aureus in 3% TSB immediately before application on gauze.

Synthetic Phenol-soluble modulin Preparation. All synthetic phenol-soluble modluins (PSM) were produced by LifeTein (Hillsborough, N.J.). Peptides were produced at 95% purity with N-terminal formylation (f). PSM sequences were as follows:

PSMaα1:
(SEQ ID NO: 5)
f-MGIIAGIIKVIKSLIEQFTGK,
PSMaα2:
(SEQ ID NO: 6)
f-MGIIAGIIKFIKGLIEKFTGK,
PSMaα3:
(SEQ ID NO: 7)
f-MEFVAKLFKFFKDLLGKFLGNN,
PSMaα4:
(SEQ ID NO: 8)
f-MAIVGTIIKIIKAIIDIFAK,
PSMβ2:
(SEQ ID NO: 9)
f-MTGLAEAIANTVQAAQQHDSVKLGTSIVDIVANGVGLLGKLFGF.

Peptides were re-suspended in DMSO and concentrated by speedvac into 500 mg powdered stocks stored at −80° C. prior to reconstitution in DMSO for experiments.

RNA isolation and quantitative real-time PCR. All RNA was isolated using the Purelink RNA isolation kit according to manufacturer's instructions (Thermo Fisher Scientific). For NHEKs, 350 μL RNA lysis buffer (with 1% β-mercaptoethanol) was added directly to cells. For mouse tissue, 0.5 cm² full thickness skin was bead beat (2×30 sec, 2.0 mm zirconia bead) in 750 μL of RNA lysis buffer with 5 minutes on ice in between. Tissue was than centrifuged (10 min, 13,000 RPM, 4° C.), followed by adding 350 μL of clear lysate to 70% EtOH and column based isolation of RNA. For S. aureus RNA isolation, 1×109 CFU bacteria was incubated with a 2:1 ratio of RNAprotect (Qiagen) for 10 min prior to centrifugation (10 min, 13,000 RPM, RT), re-suspension in 750 μL of RNA lysis buffer, and beading beating (2×1 min 6.5 speed) using lysis matrix B tubes and a Fastprep-24 (MP Biomedicals). Samples were than centrifuged again and 350 μL of clear lysate was added to 70% EtOH as above. After RNA isolation, samples were quantified with a Nanodrop (ThermoFisher Scientific), and 500 ng of RNA was reverse-transcribed using the iScript cDNA synthesis kit (Bio-Rad). qPCR reactions were ran on a CFX96 Real-Time Detection System (Bio-Rad). For mammalian cells, gene-specific primers and TaqMan probes (Thermo Fisher Scientific) were used with GAPDH as a housekeeping gene.

Generation of RP62A Competent Cells and Transformation. Electro-competent RP62A cells were prepared. Briefly, an overnight culture of S. epidermidis RP62A was diluted to an OD₆₀₀ nm of 0.5 in pre-warmed Brain Heart Infusion (BHI) broth, incubated for an additional 30 min at 37° C. with shaking, transferred to centrifuge tubes and then chilled on ice for 10 min. Cells were harvested by centrifugation (10 min, 4000 RPM, 4° C.), washed serially with 1 volume, 1/10 volume and then 1/25 volume of cold autoclaved water followed by re-pelleting at 4° C. after each wash. After the final wash, cells were re-suspended in 1/200 volume of cold 10% sterile glycerol and aliquoted at 50 μL into tubes for storage at −80° C. Transformation of S. epidermidis RP62A was carried out. Briefly, frozen competent cells were thawed on ice for 5 min and then at RT for 5 min. Thawed cells were briefly centrifuged (1 min, 5000 g, RT) and the pellet was resuspended in 50 μL of 10% glycerol supplemented with 500 mM sucrose. After addition of DNA, cells were transferred to a 1 mm cuvette and pulsed on a Micropulser (Bio-Rad) at 2.1 kV with a time constant of 1.1 msec. Immediately after electroporation, cells were re-suspended in 1 mL of BHI broth supplemented with 500 mM sucrose, shaken for 1 hr at 30° C. and then plated on BHI agar with 10 μg/mL chloramphenicol (Cm) at 30° C.

Allelic Replacement of S. epidermidis RP62A AIP. The allelic replacement plasmid pMAD (50) was used to selectively generate an in-frame deletion of the AIP coding sequence of agrD in S. epidermidis RP62A. Briefly, approximately 1000 bp fragments upstream and downstream of the AIP sequence of RP62A were amplified by PCR and joined by gene splicing by overlap extension or ‘SOEing’. The sewn fragments and pMAD vector were digested with BamHI and SalI, ligated together by T4 DNA ligase (New England Biolabs) and subsequently used to chemically transform the S. epidermidis clonal complex 10 plasmid artificial modification E. coli strain, DC10B-CC10. Transformants were plated on LB with 100 μg/mL Amp and 30 μg/mL Cm at 37° C. Correct transformants were validated by restriction digest and sequencing. The verified construct was annotated as pMAD:: ΔAIP. Electro-competent RP62A was then transformed with ˜5 μg of pMAD:: ΔAIP derived from DC10B-CC10 and then plated on BHI agar with 10 μg/mL Cm and 50 μL of 40 mg/mL 5-bromo-4-chloro-3-indolyl-β-D-galactopyranoside (X-Gal) at 30° C. A single blue colony was selected and grown in BHI with 10 μg/mL Cm overnight at 30° C. The overnight culture was then diluted 1:100 (for final volume of 100 mL) into fresh, pre-warmed, BHI without antibiotics and incubated for 24 hrs at 43° C. The dilution and growth at 43° C. was repeated an additional time to promote the single crossover event by selecting for light blue colonies grown on BHI agar supplemented with 10 μg/mL Cm and 50 μL of 40 mg/mL X-Gal at 43° C. A light blue colony was selected and incubated in BHI without antibiotics overnight at 30° C. to promote the double crossover event. Dilutions of this overnight were plated on BHI agar supplemented with 50 μL of 40 mg/mL X-Gal and incubated overnight at 37° C. White colonies were selected and patched on BHI agar supplemented with either 10 μg/mL Cm or 50 μL of 40 mg/mL X-Gal. Colonies that failed to grow in the presence of Cm and remained white in the presence of X-Gal were selected and screened for deletion of the AIP coding sequence by sequencing. The verified mutant strain was annotated as S. epidermidis RP62A ΔAIP.

RNA sequencing. RNA was submitted to the University of California, San Diego (UCSD) genomic core facility for library preparation and sequencing. TruSeq mRNA Library Prep Kit (Illumina) was used for library prep followed by high-throughput sequencing on a HiSeq 2500 sequencer (Illumina). Data was analyzed using Partek Flow and Partek Genomics Suite software and gene ontology analysis was performed using the PANTHER classification system (http[://]pantherdb.org).

Histology. Full-thickness murine skin (0.5 cm²) were collected, fixed in paraformaldehyde (4%), and washed in PBS prior to overnight incubations with 30% and 10% sucrose prior to freezing tissue in OCT mounting medium with dry ice. Cryostat cut sections (10 mm) were mounted onto Superfrost Plus glass slides (Fisher Scientific) and stained with hematoxylin and eosin (H&E). Sections were incubated for 5 min intervals in EtOH gradient of 75%-100% prior to xylene incubation and mounting with paramount and glass slide. Pictures were taken on an Olympus BX51 (Tokyo, Japan) fluorescent microscope at a 200× magnification.

Cytokine Level Determination. Conditioned medium from NHEKs (25 μL) were used to quantify protein concentration of various cytokines. Magnetic bead-based milliplex assay kits (Millipore) for 3 human cytokines (IL-6, IL-8, TNFα) were used according to the manufacture's instructions on a Magpix 200 (Luminex) system. Human IL-1α and IL-36α were quantified by ELISA (R&D Systems).

Quantification of Bacterial CFU. S. aureus colony-forming units (CFU) was quantified via plating out serial dilutions (10 μL) of 10⁻¹ to 10⁻⁵ on Baird-parker agar (BD) plates containing 3% egg yolk emulsion with tellurite for 24 h in a 37° C. incubator followed by counting the CFU. Bacterial CFU for all Staphylococci strains was also approximated using a spectrophotometer and measuring the OD600 nm of cells diluted 1:20 in PBS as well.

Transepidermal Water Loss Measurements. To determine damage to the epidermal skin barrier, transepidermal water loss (TEWL) of murine skin treated for 48-72 h with S. aureus was measured using a TEWAMETER TM300 (C & K).

Trypsin Activity Analysis. NHEK conditioned medium was added at 50 μL to black 96 well black bottom plates (Corning) followed by addition of 150 μL of the peptide Boc-Val-Pro-Arg-AMC (trypsin-like substrate; BACHEM) at a final concentration of 200 μM in 1× digestion buffer (10 mM Tris-HCl pH 7.8) and incubated at 37° C. for 24h. Relative fluorescent intensity (ex:354 nm, em:435 nm) was analyzed with a SpectraMAX Gemini EM fluorometer (Thermo Fisher Scientific). For murine skin trypsin activity analysis, 0.5 cm² full-thickness skin was bead-beat (2.0 mm zirconia beads, 2×30 sec with 5 min after each) in 1 mL of 1M acetic acid followed by an overnight rotation at 4° C. Samples were centrifuged (10 min, 13,000 RPM, 4° C.), added to a new microcentrifuge tube followed by protein concentration using a speedvac to remove all remaining acetic acid. Proteins were re-suspended in molecular grade water (500 μL) and rotated overnight at 4° C. followed by another centrifugation. Clear protein lysates were added to a new tube, and BCA (Bio-rad) analysis used to determine protein concentration. Finally, 10 μg of total protein was added to a 96 well plate followed by analysis with the trypsin substrate as above.

S. aureus agr activity. Either the S. aureus USA300 LAC agr type I P3-YFP (AH1677) or the S. aureus USA300 LAC agr type I pAmi P3-Lux (AH2759) reporter strains were used to detect S. aureus agr activity. For in vitro experiments, 1e6 CFU of S. aureus USA300 LAC agr type I P3-YFP was added to 300 μL of 3% TSB along with 100 μL of sterile-filtered commensal supernatant (25% by volume), and shaken (250 RPM) 24 h at 37° C. Bacteria was than diluted 1:20 in PBS (200 μL final) and YFP (ex:495 nm, em:530 nm) was detected using the fluorometer as above and bacterial density was determined using an OD₆₀₀ nm readout on a spectrophotometer. For murine experiments, S. aureus USA300 LAC agr type I pAmi P3-Lux activity was determined using an IVIS machine and assessing luminescent intensity after a 2 min exposure by measuring emitted photons (p/sec/cm2/sr) using the LiveImaging software (PerkinElmer).

Genome sequencing and assembling. The S. hominis C5 genomic DNA was isolated using the DNeasy UltraClean Microbial Kit (Qiagen). The libraries were sequenced using the MiSeq platform (Illumina Inc., San Diego, Calif.) for two cycles, generating 2×250 bp paired-end reads. Adapters were removed using cutadapt (version 1.9.1) (http[://]cutadapt.readthedocs.io/en/stable/). Low-quality sequences (quality score <30) were removed using the Trim Galore (version 1.9.1) (https[://][www.]bioinformatics.babraham.ac.uk/projects/trim galore/) with default parameters. Sequences mapping to the human genome were removed from the quality-trimmed dataset using the Bowtie2 (version 2.2.8) (51) with the following parameters (−D 20 −R 3 −N 1 −L 20—very-sensitive-local) and the human reference genome hg19 (UCSC Genome Browser). The filtered reads were de novo assembled using SPAdes (version 3.8.0) (52) with k-mer length ranging from 33 to 127. The genome was annotated with rapid annotation of microbial genomes using subsystems technology (RAST) with default parameters. Amino acid sequences from annotated CDS (coding DNA sequence) were aligned to bacterial agr proteins obtained from Uniprot database (downloaded in October 2017). Agr genes from the assembled genome were identified following three criteria: i) sequence identity >60%; ii) e-value <e100; and iii) the agr locus organization, an operon of four genes, agrBDCA.

Microbiome data and genome comparative analysis. Publicly available shotgun metagenomic data for atopic dermatitis skin was analyzed. Relative abundance of S. aureus and S. epidermidis strains were obtained directly from the published supplementary material ([www.]sciencetranslationalmedicine.org/cgi/content/full/9/397/eaa14651/DC1). The agrD characterization analysis was restricted to eight patients (AD01, AD02, AD03, AD04, AD05, AD08, AD09, and AD11) with information at 7 distinct body sites on flared AD skin and differences in AD severity based upon objective SCORAD. The 61 S. epidermidis strains evaluated were classified as agr type I, II, or III through amino acid sequence comparison with known agr type I-III sequences within the agrD gene region.

Quantification and Statistical Analysis. The nonparametric Mann-Whitney test was used for statistical significance analysis of AD patient metagenomic data. Either One-way ANOVA or Two-way ANOVA for statistical analysis as indicated in the various figure descriptions. All statistical analysis was performed using GraphPad Prism Version 6.0 (GraphPad, La Jolla, Calif.). All data is presented as mean±standard error of the mean (SEM) and a P-value 0.05 considered significant.

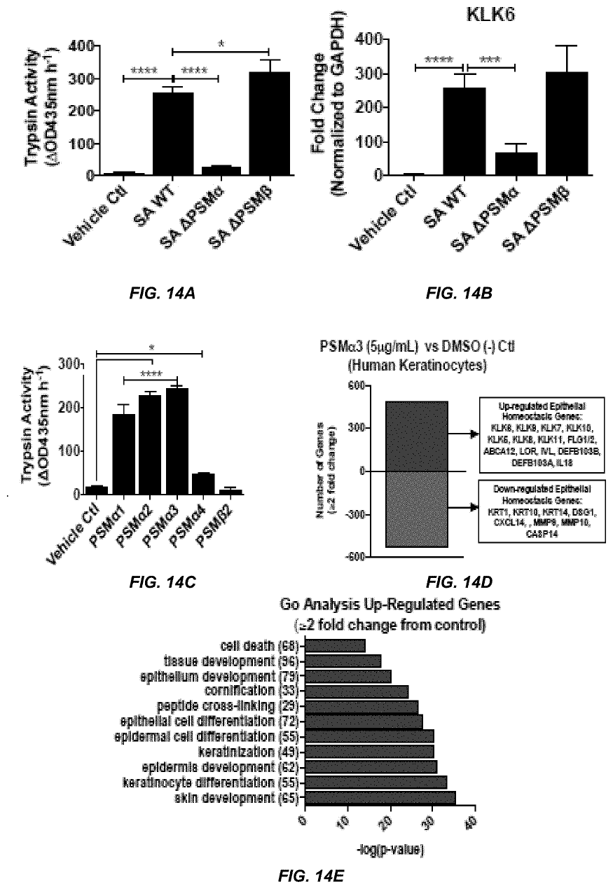

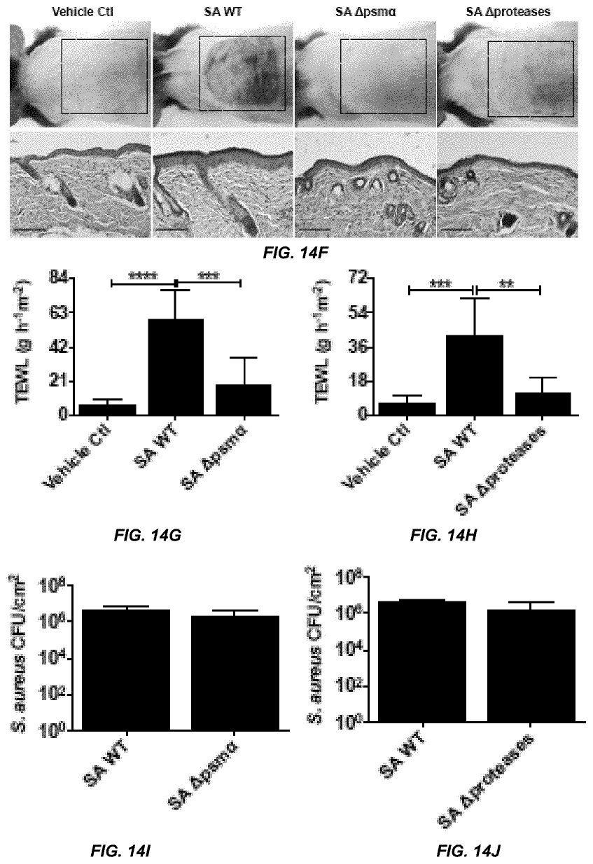

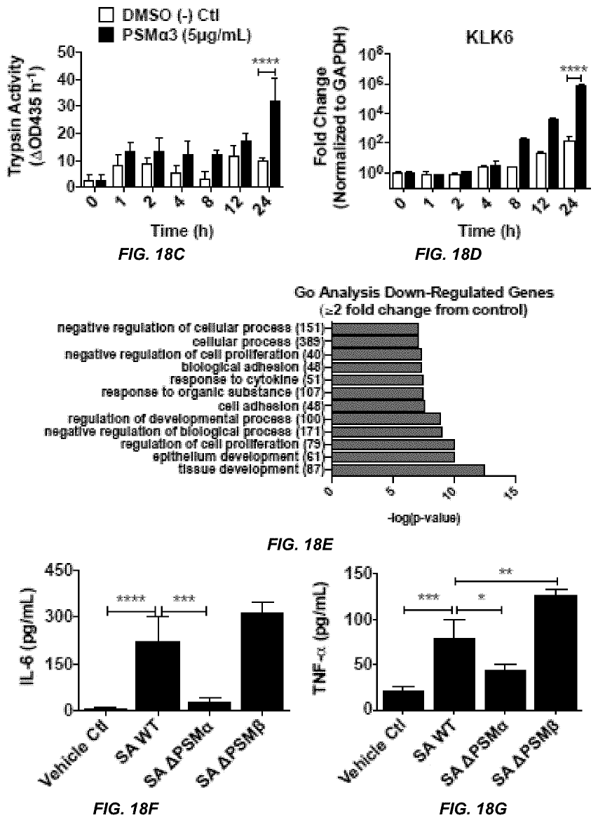

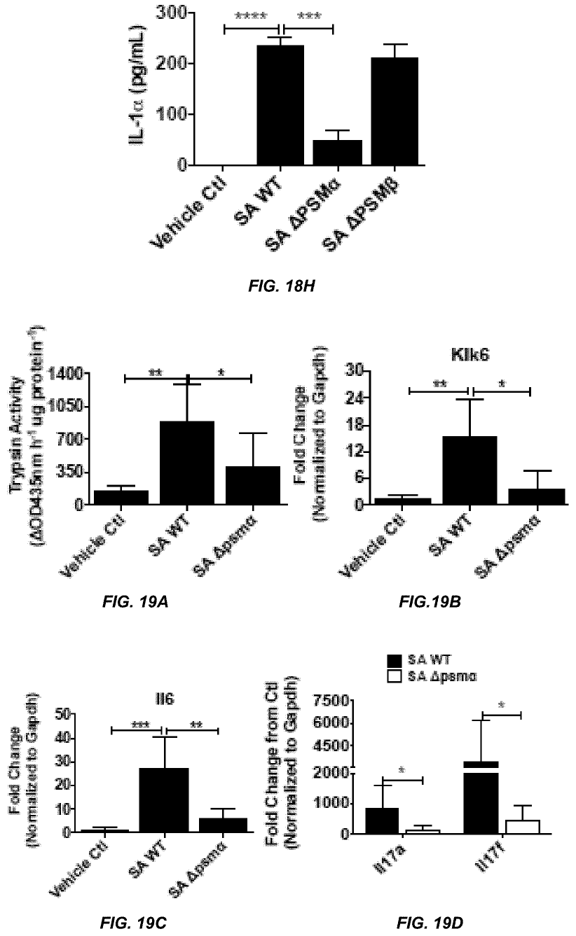

PSMα and proteases produced by S. aureus induce epidermal barrier damage. A primary function of human skin is to establish a physical barrier against the external environment. Specific toxins produced by S. aureus such as the phenol soluble modulins (PSM) can promote epithelial inflammation and have been proposed to be key to driving disease in AD(19-22). Therefore, to understand how S. aureus on the skin surface could influence inflammatory activity across the epidermal barrier normal human epidermal keratinocytes (NHEK) were assessed for their capacity to expresses proteolytic activity when exposed to a S. aureus USA300 LAC strain that has a targeted deletion in either the PSMα or PSMb operons. PSMα production was required for induction of trypsin-like serine protease activity and increased mRNA levels of kallikrein 6 (KLK6) (FIG. 14A-B), The PSMα and PSM operons in S. aureus contain distinct peptides including PSMα1-4 and PSMβ1-2. Thus synthetic PSMα1-4 and PSMβ2 peptides were tested on NHEKs and found that (FIG. 14C) all PSMα peptides could stimulate trypsin activity while PSMβ2 could not. PSMα3, the strongest PSMα trypsin activity inducers in NHEKs, was selected to further show it could stimulate trypsin activity and KLK6 mRNA expression in NHEKs in both a dose and time dependent manner (FIG. 18). Furthermore, transcriptional profiling by RNA-Seq of NHEKs exposed to PSMα3 showed this toxin had a broad effect on expression of genes related to the skin barrier including multiple proteases (KLKs, MMPs), components of the physical barrier (filaggrin, desmoglein-1, loricrin, involucrin, keratins), antimicrobial peptides, and cytokines (FIG. 14D-E, FIG. 18).

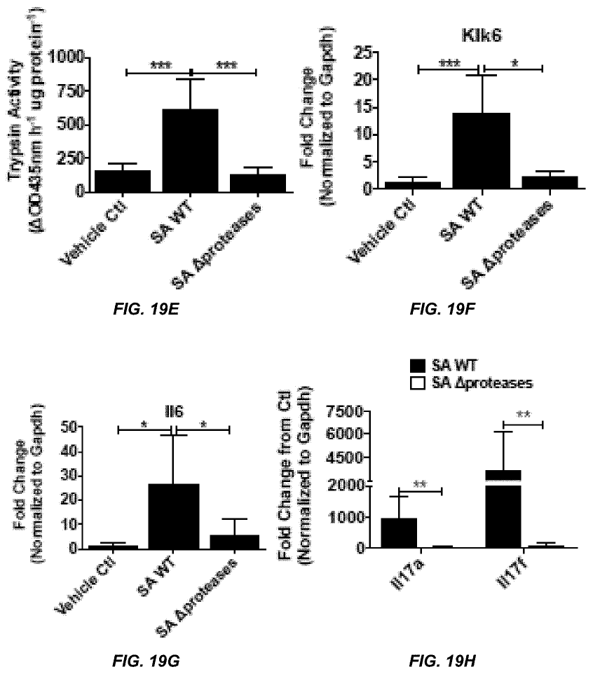

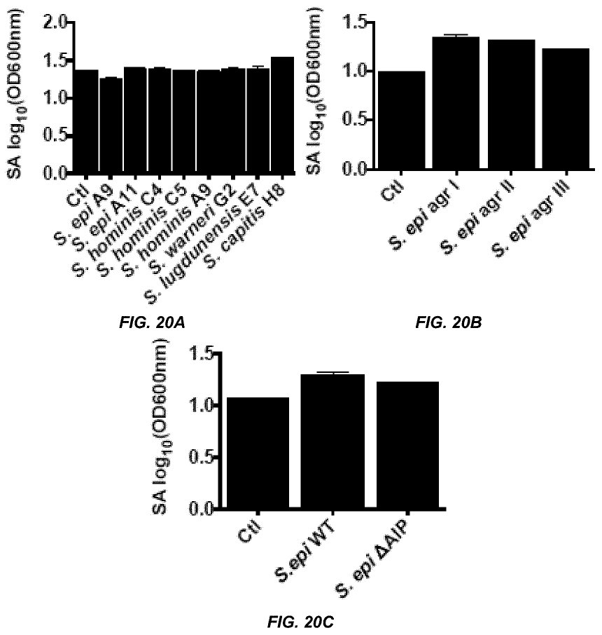

To validate the role of the PSMα operon on the epidermal barrier in vivo, mice were colonized for 72 h on the skin surface with equal numbers of the S. aureus USA300 LAC wild type or the PSMα mutant strain. Wild type S. aureus induced erythema, scaling, and epidermal thickening while no change in bacterial abundance was observed in the absence of PSMα (FIG. 14F). Despite increased epidermal thickness, an increase in transepidermal water loss (TEWL), a well-established method to assess skin barrier damage, was observed after exposure to wild-type S. aureus but not when PSMα was absent (FIG. 14G). However, skin barrier disruption of a fully differentiated epidermis in vivo was also dependent on S. aureus protease expression. Using a S. aureus USA300 LAC mutant strain that lacks 10 major secreted proteases including aureolysin, V8, staphopain A/B, and SplA-F, visible evidence of injury and increased TEWL was diminished in a S. aureus protease deficient manner despite fully intact PSMα expression (FIG. 14F,H). Coincident with the gross and histologic changes observed to be associated with expression of either PSMα or bacterial proteases, an increase in keratinocyte trypsin activity, Klk6 transcript expression and cytokines 116, 1117a, and 1117f was measured only in mice exposed to wild-type S. aureus but not in PSMα or protease deficient strains (FIG. 19). Furthermore, despite changes in the skin barrier and inflammatory milieu of the skin, S. aureus abundance was unchanged on the skin surface under these conditions (FIG. 14I-J). Taken together, these data suggest that production of PSMα and protease activity from S. aureus results in damage to the epidermal barrier and that this barrier damage is required for S. aureus to promote inflammation.

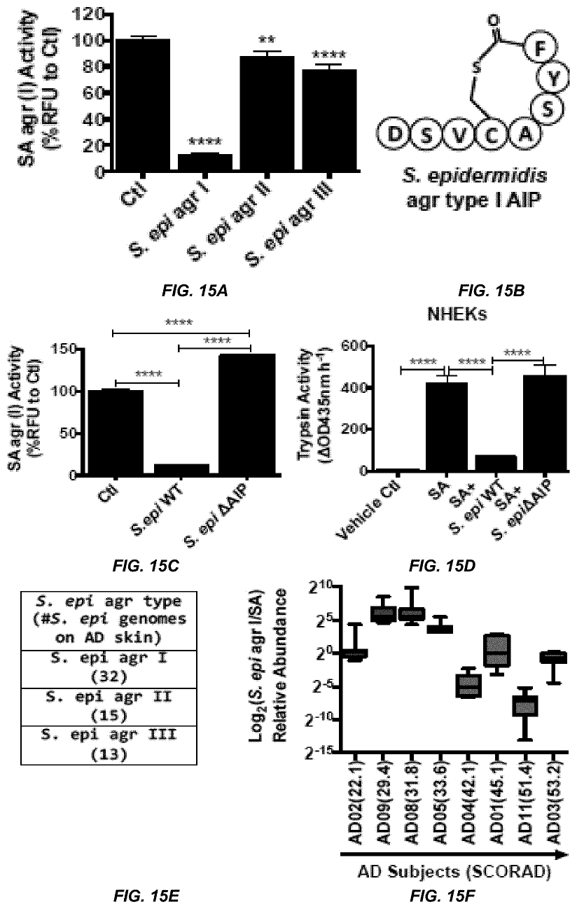

S. epidermidis auto-inducing peptide inhibits S. aureus agr activity. Interestingly, both the S. aureus PSMα peptides and secreted proteases are under regulation of the agr quorum sensing system. S. aureus clinical isolates furthermore have been found to have four distinct agr types with agr type I being the most prominent in AD subjects. Although S. aureus skin colonization increases in AD, other bacterial species such as coagulase-negative Staphylococci (CoNS) strains including the abundant human skin commensal organism S. epidermidis are also present making it essential to understand how these bacteria communicate. S. epidermidis agr type I lab isolates have been shown to produce an autoinducing peptide (AIP) that to inhibits the S. aureus agr type I-III systems but not type IV through an agr crosstalk mechanism, while little is known of the other S. epidermidis agr types II and III on their influence on S. aureus agr activity. Conditioned culture supernatants from S. epidermidis strains with agr types I, II, or III were added to a S. aureus USA300 LAC agr type I reporter strain to explore if S. epidermidis agr activity could influence the S. aureus agr system. This experiment confirmed that S. epidermidis agr I was the only potent inhibitor of S. aureus agr activity while S. epidermidis agr type II and III had little effect (FIG. 15A). Targeted deletion of the S. epidermidis agr type I AIP within the agrD gene region abolished the capacity of S. epidermidis to inhibit S. aureus agr activity (FIG. 15B-C). Since S. aureus PSMα induced NHEK trypsin activity is a component to epidermal barrier damage, S. epidermidis agr type I wild type or AIP knockout strain were tested to determine if they could effect this result. It was observed that S. aureus induced NHEK trypsin activity was inhibited when S. aureus was cultured in the presence of wild type S. epidermidis agr type I supernatant but not by S. epidermidis lacking this AIP (FIG. 15D). Overall, these experiments established that S. aureus capacity to induce NHEK barrier damage can be influenced by S. epidermidis agr type I AIP expression.

Deficiency in S. epidermidis agr type I relative abundance on AD skin. Having established the potential for a laboratory strain of S. epidermidis to influence the effects of S. aureus on the function of human keratinocytes, experiments were performed to determine the abundance of these bacteria in a clinical setting. Metagenomic data available from the skin microbiome of 8 subjects with AD of different severity (based upon the objective SCORAD) collected from 7 body sites were analyzed for S. epidermidis relative abundance based upon agr type. Sequence alignments identified S. epidermidis genomes based on agr types IIII on AD patients and determined the most frequent S. epidermidis agr type on AD skin is that of agr type I (FIG. 15E). Comparison of S. epidermidis agr I to S. aureus showed that S. epidermidis agr type I became relatively less abundant in AD subjects with increased disease severity (FIG. 15F-G). These observations confirmed the presence of S. epidermidis agr type I in the AD skin microbiome and suggest the potential for association with clinical disease.

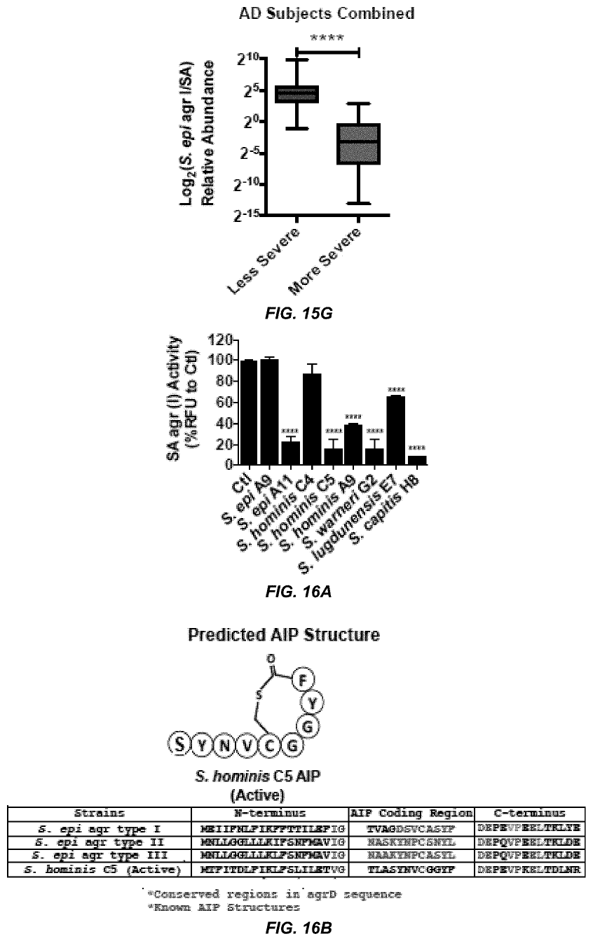

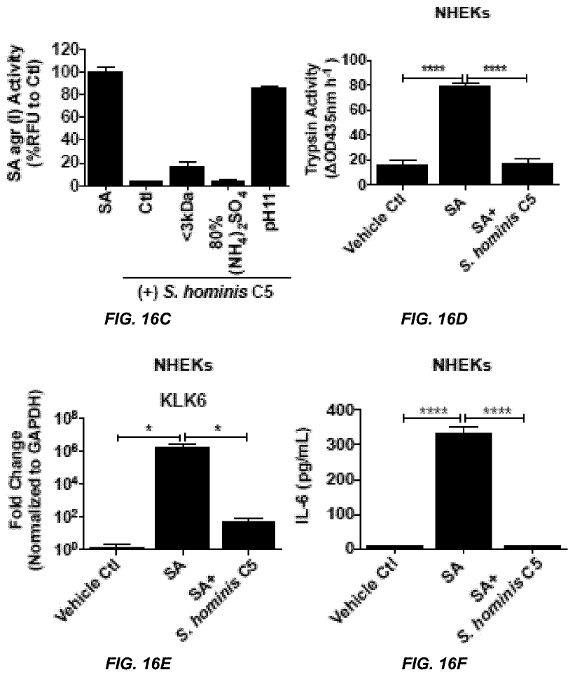

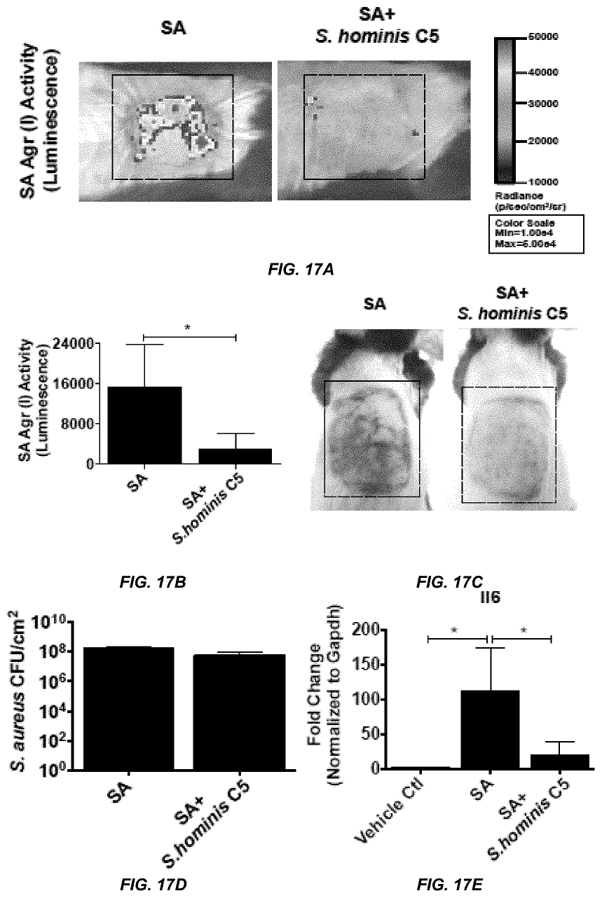

Diverse Staphylococci species and strains inhibit S. aureus agr activity. To further establish the physiological significance of quorum sensing interactions between S. aureus and other members of the skin microbiome, different AD clinical isolates of CoNS were tested for the capacity of their culture supernatant to inhibit S. aureus USA300 LAC agr type I quorum sensing activity. Diverse species including S. epidermidis, S. hominis, S warneri, and S capitis showed potent inhibitory activity against agr activity of S. aureus (FIG. 16A). Similar to the lab isolates of S. epidermidis, the CoNS strains inhibited S. aureus agr activity without inhibiting the growth rate (FIG. 316S). Furthermore, a genomic sequence analysis of the agrD AIP coding region of S. hominis strain C5 revealed a novel AIP sequence in the AIP coding region similar to the sequence of S. epidermidis agr type I coding region and with a predicted octomer AIP sequence for S. hominis C5 (FIG. 16B; SEQ ID NO:4). Biochemical techniques of the active S. hominis C5 supernatant showed that inhibition of S. aureus agr activity was dependent on a <3 kDa (small size), pH 11 sensitive (thiolactone ring) factor that could be precipitated with 80% ammonium sulfate (peptide) (FIG. 16C).

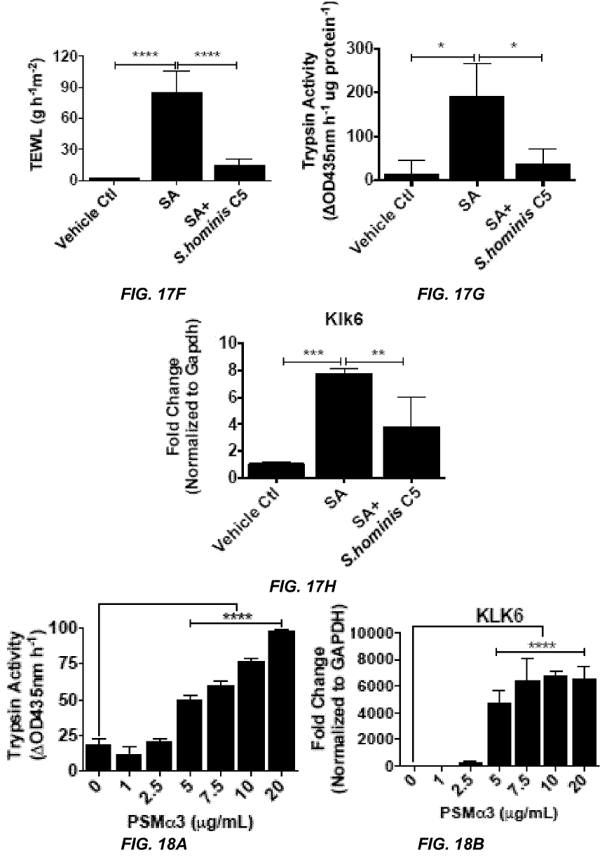

Next, S. aureus was cultured in the presence of S. hominis C5 sterile-filtered supernatant and the subsequent culture supernatant was applied to NHEKs as in FIG. 14. Similar to S. epidermidis agr type I, S. hominis C5 inhibited S. aureus induced trypsin activity, KLK6 transcript production, and IL-6 protein expression in NHEKs (FIG. 16D-F). Furthermore, S. hominis C5 could inhibit multiple S. aureus agr systems aside from most common clinical isolate of agr type I including agr types II and III but not IV (FIG. 17S). This finding coincides with what has been observed with the S. epidermidis agr type I system. Overall, these observations suggest clinical isolates of CoNS species in addition to S. epidermidis may use quorum sensing to suppress S. aureus damage to keratinocytes.

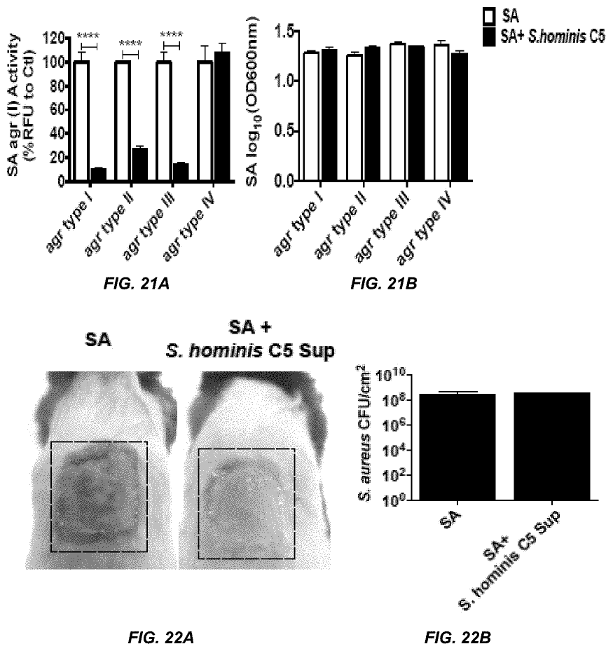

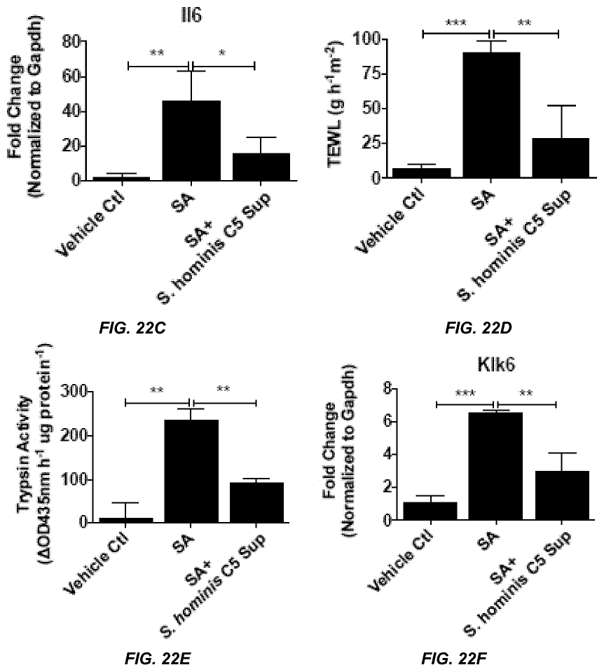

A clinical CoNS isolate inhibits S. aureus agr activity its ability to promote AD. To establish the physiological relevance of quorum sensing interactions between CoNS and S. aureus in vivo, S. aureus agr activity was assessed by IVIS using a S. aureus USA300 LAC agr type I P3-Lux promoter (luminescence) strain. S. aureus on back skin showed abundant agr activity but when in the presence of live S. hominis C5, S. aureus agr activity was inhibited (FIG. 17A-B). Furthermore, S. hominis C5 also protected against S. aureus induced skin erythema and scaling (FIG. 17C) without altering S. aureus abundance (FIG. 17D). This phenotype was associated with improved evidence for inflammation, barrier disruption, and epidermal protease activity and Klk6 expression (FIG. 17E-H). Furthermore, when S. aureus was applied to murine back skin in the presence of a <3 kDa concentrated S. hominis C5 supernatant, similar reductions in barrier damage and inflammation were observed without changes to S. aureus abundance. (FIG. 22). These data show the skin CoNS microbial community likely contains novel AIPs that promote epithelial barrier homeostasis by interspecies quorum sensing activity.

A number of embodiments of the disclosure have been described. Nevertheless, it will be understood that various modifications may be made without departing from the spirit and scope of the disclosure. Accordingly, other embodiments are within the scope of the following claims.

1-21. (canceled)

22. A method of treating a dermatological disorder comprising administering an effective amount of at least one coagulase negative Staphylococcus sp. (CoNS), or an effective amount of a fermentation extract of CoNS sufficient to inhibit protease activity on the skin, wherein the CoNS produces a polypeptide comprising a sequence that is at least 98% identical to SEQ ID NO:4, 11, 12, 13, 14, 15, 16, or 17 and which inhibits protease production.

23. The method of claim 22, wherein the dermatological disorders is selected from the group consisting of Netherton syndrome, atopic dermatitis, contact dermatitis, eczema, psoriasis, acne, epidermal hyperkeratosis, acanthosis, epidermal inflammation, dermal inflammation and pruritus.

24. The method of claim 22, wherein the administering is by topical application.

25. The method of claim 22, wherein the at least one CoNS is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus Hominis, Staphylococcus jettensis, Staphylococcus petrasii, and Staphylococcus lugdunensis.

26. The method of claim 22, wherein the fermentation extract of the CoNS comprises a polypeptide sequence of SEQ ID NO:4, 11, 12, 13, 14, 15, 16 or 17 and/or a compound of Formula I.

27. The method of claim 22, wherein the at least one CoNS is selected from the group consisting of S. epidermidis A11, S. hominis C4, S. hominis C5, S. hominis A9, S. warneri G2 and any combination thereof.

28. A method of treating a skin disease or disorder, comprising
measuring the protease activity of a culture from skin of a subject or of skin from the subject;
comparing the protease activity to a normal control;
administering a commensal skin bacterial composition and/or fermentation extract from a coagulase negative Staphylococci, wherein the commensal skin bacteria composition or fermentation extract comprises a polypeptide that is at least 98% identical to SEQ ID NO:4, 11, 12, 13, 14, 15, 16 or 17 and/or comprises a compound of Formula I, wherein the composition is formulated in a cream, ointment or pharmaceutical composition that maintain the commensal skin bacteria's ability to grow and replicate.

29. The method of claim 28, wherein the coagulase negative Staphylococci is selected from the group consisting of is Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus Hominis, Staphylococcus jettensis, Staphylococcus petrasii, and Staphylococcus lugdunensis.

30. A method of treating a skin disease or disorder comprising administering a probiotic composition comprising a bacteria that produces a polypeptide that is at least 95% identical to SEQ ID NO:4, 11, 12, 13, 14, 15, 16 or 17 and which inhibits (i) protease production and/or activity of keratinocytes, (ii) inhibits IL-6 production and/or activity of keratinocytes, (iii) inhibits production of phenol soluble modulin alpha 3 from Staphylococcus aureus (S. aureus) and/or (iv) inhibits adr production and/or activity by S. aureus.

31. (canceled)

32. The method of claim 30, wherein the administering is topical.

33. (canceled)

34. The method of claim 30, wherein the probiotic composition comprises at least one probiotic commensal skin bacteria selected from the group consisting of S. epidermidis A11, S. hominis C4, S. hominis C5, S. hominis A9, S. warneri G2 and any combination thereof.

35. The method of claim 30, wherein the probiotic composition is formulated as a lotion, shake lotion, cream, ointment, gel, foam, powder, solid, paste or tincture.

36-37. (canceled)

38. The method of claim 22, wherein the administering is topical, and wherein the at least one CoNS and/or the fermentation extract of the CoNS is formulated as a lotion, shake lotion, cream, ointment, gel, foam, powder, solid, paste or tincture.

39. The method of claim 22, wherein the polypeptide inhibits (i) protease production and/or activity of keratinocytes, (ii) inhibits IL-6 production and/or activity of keratinocytes, (iii) inhibits production of phenol soluble modulin alpha 3 from Staphylococcus aureus (S. aureus) and/or (iv) inhibits agr production and/or activity by S. aureus.

40. The method of claim 28, wherein the skin disease or disorder is selected from the group consisting of Netherton syndrome, atopic dermatitis, contact dermatitis, eczema, psoriasis, acne, epidermal hyperkeratosis, acanthosis, epidermal inflammation, dermal inflammation and pruritus.

41. The method of claim 28, wherein the administering is by topical application.

42. The method of claim 28, the coagulase negative Staphylococci is selected from the group of microorganisms having ATCC Number PTA-125202 (strain designation S. epidermidis A11 81618, deposited Aug. 28, 2018), ATCC Number PTA-125204 (strain designation S. hominis C5 81618, deposited Aug. 28, 2018), ATCC Number PTA-125203 (strain designation S. hominis A9 81618, deposited Aug. 28, 2018), ATCC Number PTA-125205 (strain designation S. warneri G2 81618, deposited Aug. 28, 2018) and any combination of the foregoing strains.

43. The method of claim 28, wherein the polypeptide inhibits (i) protease production and/or activity of keratinocytes, (ii) inhibits IL-6 production and/or activity of keratinocytes, (iii) inhibits production of phenol soluble modulin alpha 3 from Staphylococcus aureus (S. aureus) and/or (iv) inhibits agr production and/or activity by S. aureus.

44. The method of claim 30, wherein the polypeptide inhibits kallikrein expression and/or phenol soluble modulin expression.

45. The method of claim 30, wherein the skin disease or disorder is selected from the group consisting of Netherton syndrome, atopic dermatitis, contact dermatitis, eczema, psoriasis, acne, epidermal hyperkeratosis, acanthosis, epidermal inflammation, dermal inflammation and pruritus.


SEQUENCE LISTING (17 sequences)

SEQ ID NO: 1 - DNA, 141 bp, Staphylococcus hominis
  CDS (1)..(141)
atgacatttattacagatttattcattaaactattctcattaatcttagaaactgttggtacacttgcttcatataatgtatgtggtggttatttcgatgaacctgaagttcctaaagaattaactgatcttaatagataa

SEQ ID NO: 2 - protein, 46 aa, Staphylococcus hominis
MTFITDLFIKLFSLILETVGTLASYNVCGGYFDEPEVPKELTDLNR

SEQ ID NO: 3 - DNA, 27 bp, Artificial Sequence
  Coding fragment from S. hominis
  CDS (1)..(27)
tcatataatgtatgtggtggttatttc

SEQ ID NO: 4 - protein, 9 aa, Artificial Sequence
  Synthetic Construct
SYNVCGGYF

SEQ ID NO: 5 - protein, 21 aa, Artificial Sequence
  Phenol Soluble Modulin alpha-1
MGIIAGIIKVIKSLIEQFTGK

SEQ ID NO: 6 - protein, 21 aa, Artificial Sequence
  Phenol Soluble Modulin alpha-2
MGIIAGIIKFIKGLIEKFTGK

SEQ ID NO: 7 - protein, 22 aa, Artificial Sequence
  Phenol Soluble Modulin alpha-3
MEFVAKLFKFFKDLLGKFLGNN

SEQ ID NO: 8 - protein, 20 aa, Artificial Sequence
  Phenol Soluble Modulin alpha-4
MAIVGTIIKIIKAIIDIFAK

SEQ ID NO: 9 - protein, 44 aa, Artificial Sequence
  Phenol Soluble Modulin beta-2
MTGLAEAIANTVQAAQQHDSVKLGTSIVDIVANGVGLLGKLFGF

SEQ ID NO: 10 - protein, 9 aa, Artificial Sequence
  AIP consensus sequence
  MISC_FEATURE (1)..(1): Xaa is S, K, V, G or T
  MISC_FEATURE (2)..(2): Xaa is Y, Q, A, or I
  MISC_FEATURE (3)..(3): Xaa is N, S, T, or D
  MISC_FEATURE (4)..(4): Xaa is V, P, M, or T
  MISC_FEATURE (6)..(6): Xaa is G, S, A, N, or T
  MISC_FEATURE (7)..(7): Xaa is G, N, T, or L
  MISC_FEATURE (8)..(8): Xaa is Y or F
  MISC_FEATURE (9)..(9): Xaa is Y, F or L
XXXXCXXXX

SEQ ID NO: 11 - protein, 9 aa, Artificial Sequence
  AIP peptide from S. epidermidis strain A11
KYNPCSNYL

SEQ ID NO: 12 - protein, 9 aa, Artificial Sequence
  AIP peptide from S. hominis strains A9 and C4
SYSPCATYF

SEQ ID NO: 13 - protein, 9 aa, Artificial Sequence
  AIP peptide from S. hominis
SQTVCSGYF

SEQ ID NO: 14 - protein, 9 aa, Artificial Sequence
  AIP peptide from S. hominis and S. capitis
GANPCALYY

SEQ ID NO: 15 - protein, 9 aa, Artificial Sequence
  AIP peptide from S. hominis
TINTCGGYF

SEQ ID NO: 16 - protein, 9 aa, Artificial Sequence
  AIP peptide from S. lugdunensis
VQDMCNGYF

SEQ ID NO: 17 - protein, 9 aa, Artificial Sequence
  AIP peptide from S. warneri strain G2
GYSPCTNFF